SEQUENCE LISTING (14 sequences)

SEQ ID NO: 1 - DNA, 18 bp, Artificial sequence
  Synthesized
gcatccactggtgctgcc

SEQ ID NO: 2 - DNA, 21 bp, Artificial sequence
  Synthesized
tcatcatacttggcaggtttc

SEQ ID NO: 3 - DNA, 20 bp, Artificial sequence
  Synthesized
tagccaggagggagaacaga

SEQ ID NO: 4 - DNA, 20 bp, Artificial sequence
  Synthesized
ttttctggagggagatgtgg

SEQ ID NO: 5 - DNA, 20 bp, Artificial sequence
  Synthesized
ttgaagaagagcccatcctc

SEQ ID NO: 6 - DNA, 19 bp, Artificial sequence
  Synthesized
cagctcatatgggtccgac

SEQ ID NO: 7 - DNA, 27 bp, Artificial sequence
  Synthesized
ttacgatggaattctccttgtatatca

SEQ ID NO: 8 - DNA, 21 bp, Artificial sequence
  Synthesized
cacagcaggaaggtggctatg

SEQ ID NO: 9 - DNA, 25 bp, Artificial sequence
  Synthesized
cagtcacgtgctgttataatgttgt

SEQ ID NO: 10 - DNA, 25 bp, Artificial sequence
  Synthesized
tatggaattcttaacccacttctcc

SEQ ID NO: 11 - DNA, 24 bp, Artificial sequence
  Synthesized
acccgaaactgatgctgtggatag

SEQ ID NO: 12 - DNA, 24 bp, Artificial sequence
  Synthesized
aaatggccgggcagaacttgtgta

SEQ ID NO: 13 - DNA, 19 bp, Artificial sequence
  Synthesized
atgtccggccgatgctctc

SEQ ID NO: 14 - DNA, 23 bp, Artificial sequence
  Synthesized
tttggctgctcttgggtctgtat

(19) United States
(12) Patent Application Publication
(10) Pub. No.: US 2022/0175821 A1
(43) Pub. Date: Jun. 9, 2022

(54) METHODS FOR IMPROVING ANTI-OXIDATION AND PREVENTING/TREATING DISEASES USING HYPO-ACYLATED LIPOPOLYSACCHARIDE

(71) Applicant: Multistars Biotechnology Company Limited, Taoyuan City (TW)

(72) Inventors: HSIN-CHIH LAI, Taoyuan City (TW); CHIA-CHEN LU, Taoyuan City (TW); TZU-LUNG LIN, Taoyuan City (TW); CHUN-HUNG LIN, Taoyuan City (TW); CHENG-I DANIEL YAO, Taoyuan City (TW); PO-I WU, Taoyuan City (TW)

(21) Appl. No.: 17/511,888

(22) Filed: Oct. 27, 2021

Related U.S. Application Data
(60) Provisional application No. 63/106,110, filed on Oct. 27, 2020.

(51) Int. Cl. A61K 31/739; A23L 33/10; A61P 11/00; A61P 3/08; A61P 3/04

(52) U.S. Cl. CPC A61K 31/739; A23L 33/10; A61P 3/04; A61P 3/08; A61P 11/00

(57) ABSTRACT

The present invention provides methods of hypo-acylated lipopolysaccharide (LPS) for improving anti-oxidation and preventing/treating endotoxemia and diseases associated with endotoxemia.

### CROSS-REFERENCE TO RELATED APPLICATION

This application claims priority of U.S. provisional application No. 63/106,110, filed on Oct. 27, 2020, the content of which are incorporated herein in its entirety by reference.

### BACKGROUND OF THE INVENTION

### 1. Field of the Invention

The present invention relates to methods for improving anti-oxidation and preventing/treating diseases using lipopolysaccharide, and more particularly to methods for improving anti-oxidation, preventing and/or treating endotoxemia, preventing and/or treating chronic obstructive pulmonary disease, preventing and/or treating obesity, and increasing glucose tolerance using lipopolysaccharide with hypo-acylated lipid A structure.

### 2. The Prior Art

Lipopolysaccharide (LPS) is one of the main components on the cell membrane of Gram-negative bacteria, and it is also a marker of bacterial invasion and is a kind of endotoxin. Lipopolysaccharide mainly provides and maintains the structural integrity of bacteria, and protects the cell membrane of bacteria against attack of certain chemicals, such as the immune response from the host. When microorganisms invade individuals and release lipopolysaccharides, they would stimulate immune cells to secrete cytokines that promote inflammation, such as tumor necrosis factor-α (TNF-α) and interleukin-1 (Interleukin-1, IL-1), etc., and cause the individuals to produce inflammatory responses, and could even lead to the occurrence of sepsis, and the most serious may be fatal.

The health of intestine is closely related to various physiological systems of individuals. Leaky gut syndrome (LGS) refers to inflammation and destruction of the intestinal mucosa causing leaky between cells of the intestinal mucosa, and then making the intestinal substances leak into the blood and lymph from the intercellular space to induce adverse reactions such as systemic low-grade inflammation. If this adverse reaction is not controlled and leads to a whole-body imbalance, it will further affect the health of the intestine, so that the barrier function of the intestinal mucosal cells will continue to be damaged, and the intestinal leakage will continue to occur and form a vicious circle. The leakage of bacterial lipopolysaccharide from the intestine could lead to the occurrence of endotoxemia, and therefore adversely affect the health of different organs and tissues.

However, there is still a lack of clinically safe and effective methods for the treatment of endotoxemia. Currently, blood purification methods are used to reduce endotoxin levels in the blood. However, blood purification methods require direct contact with blood of the individuals. Therefore, it may cause adverse reactions such as affecting plasma components, causing electrolyte imbalance, destroying the enzyme system, causing harmful immune and allergic reactions, carcinogenicity, and causing hemolytic reactions.

Therefore, it is really necessary to develop a safe and effective composition or method to reduce the harm of bacterial lipopolysaccharide to individuals.

### SUMMARY OF THE INVENTION

To solve the foregoing problem, one objective of the present invention is to provide a method for improving anti-oxidation, comprising administering to a subject in need thereof a composition comprising a hypo-acylated lipopolysaccharide, wherein a lipid A of the hypo-acylated lipopolysaccharide contains 1 to 5 fluorenyl chain(s).

In one embodiment of the present invention, the hypo-acylated lipopolysaccharide promotes glutathione biosynthetic process, cell redox homeostasis, hydrogen peroxide catabolic process, sulfur compound biosynthetic process, response to oxygen-containing compound, or any combination thereof.

The further objective of the present invention is to provide a method of preventing and/or treating endotoxemia and a disease associated with endotoxemia, comprising administering to a subject in need thereof a composition comprising a hypo-acylated lipopolysaccharide, wherein a lipid A of the hypo-acylated lipopolysaccharide contains 1 to 5 fluorenyl chain(s).

In one embodiment of the present invention, the endotoxemia and the disease associated with endotoxemia is caused by leaky gut syndrome (LGS).

In one embodiment of the present invention, the hypo-acylated lipopolysaccharide promotes intestinal integrity of the subject in need thereof or reduces intestinal inflammation of the subject in need thereof.

In one embodiment of the present invention, the hypo-acylated lipopolysaccharide reduces the amount of endotoxin in blood of the subject in need thereof.

In one embodiment of the present invention, the disease associated with endotoxemia is selected from the group consisting of liver cirrhosis, primary biliary cholangitis, nonalcoholic fatty liver disease, obesity, type II diabetes, active Crohn's disease, ulcerative colitis, severe acute pancreatitis, obstructive jaundice, chronic heart failure, chronic kidney disease, chronic obstructive pulmonary disease, depression, autism, Alzheimer's disease/dementia, Parkinson disease, Huntington disease, psoriasis, atopic dermatitis, cancer, asthma, and ageing thereof.

In one embodiment of the present invention, the cancer is carcinoma, sarcoma, myeloma, leukemia, lymphoma, or mixed type tumor.

Another objective of the present invention is to provide a method of preventing and/or treating chronic obstructive pulmonary disease, comprising administering to a subject in need thereof a composition comprising a hypo-acylated lipopolysaccharide, wherein a lipid A of the hypo-acylated lipopolysaccharide contains 1 to 5 fluorenyl chain(s).

In one embodiment of the present invention, the hypo-acylated lipopolysaccharide improves body weight loss, abnormal lung function, infiltration of immune cell in lung, emphysema, secretion of pro-inflammatory cytokines, or increase in circulating endotoxin levels caused by chronic obstructive pulmonary disease.

In one embodiment of the present invention, the pro-inflammatory cytokines includes tumor necrosis factor-α (TNF-α) or interleukin-1p (IL-1β).

Another objective of the present invention is to provide a method of preventing and/or treating obesity, comprising administering to a subject in need thereof a composition comprising a hypo-acylated lipopolysaccharide, wherein a lipid A of the hypo-acylated lipopolysaccharide contains 1 to 5 fluorenyl chain(s).

In one embodiment of the present invention, the hypo-acylated lipopolysaccharide reduces increase in body weight of the subject in need thereof.

The other objective of the present invention is to provide a method of increasing glucose tolerance, comprising administering to a subject in need thereof a composition comprising a hypo-acylated lipopolysaccharide, wherein a lipid A of the hypo-acylated lipopolysaccharide contains 1 to 5 fluorenyl chain(s).

In one embodiment of the present invention, an effective amount of the hypo-acylated lipopolysaccharide is 10 μg/kg for the subject in need thereof at least twice a week.

In one embodiment of the present invention, the hypo-acylated lipopolysaccharide is a hypo-acylated lipopolysaccharide from a bacterium of Bacteroidetes.

In one embodiment of the present invention, the hypo-acylated lipopolysaccharide is a hypo-acylated lipopolysaccharide from a bacterium of Bacteroide and/or Parabacteroide.

In one embodiment of the present invention, the composition further comprises a pharmaceutically acceptable excipient, carrier, adjuvant, or food additive.

In one embodiment of the present invention, the composition is in the form of a spray, a solution, a semi-solid preparation, a solid preparation, a gelatin capsule, a soft capsule, a tablet, a chewing gum, or a freeze-dried powder preparation.

The present invention proves that the lipopolysaccharides with the structure of hypo-acylated lipid A contains low immune-stimulatory responses itself, and provides low endotoxicity to individuals, and can antagonize the immune responses induced by pathogenic lipopolysaccharides; moreover, the lipopolysaccharides with the structure of hypo-acylated lipid A can further promote antioxidant responses of cells, prevent and/or treat endotoxemia, and also prevent and/or treat diseases caused by pathogenic lipopolysaccharide or endotoxin, including but not limited to prevention/treatment of chronic obstructive pulmonary disease, prevention and/or treatment of obesity, and increasing of glucose tolerance.

The embodiments of the present invention are further described with the following drawings. The following embodiments are given to illustrate the present invention and are not intended to limit the scope of the present invention, and one with ordinary skill in the art can make some modifications and refinements without departing from the spirit and scope of the present invention. Therefore, the scope of the present invention is defined by the scope of the appended claims.

### BRIEF DESCRIPTION OF THE DRAWINGS

FIG. 1 shows the reaction formula of the biochemical synthesis pathway of Kdo₂-lipid in Escherichia coli (E. coli).

FIG. 2A shows a mass spectrometric analysis result of lipopolysaccharide of Bacteroides fragilis (B. fragilis) according to one embodiment of the present invention.

FIG. 2B shows a mass spectrometric analysis result of lipopolysaccharide of Parabacteroides goldsteinii (P. goldsteinii) according to one embodiment of the present invention.

FIG. 3A shows induced NF-κB activities of HEK-Blue-mTLR4 reporter cells after being treated with lipopolysaccharides of E. coli, P. goldsteinii, Parabacteroide distasonis (P. distasonis), Parabacteroide merdae (P. merdae), B. fragilis, or Bacteroides ovatus (B. ovatus); wherein, Ec-LPS represents the comparative group treated with lipopolysaccharide of E. coli O111:B4; Pg-LPS represents the experimental group treated with lipopolysaccharide of P. goldsteinii; Pd-LPS represents the experimental group treated with lipopolysaccharide of P. distasonis; Pm-LPS represents the experimental group treated with lipopolysaccharide of P. merdae; Bf-LPS represents the experimental group treated with lipopolysaccharide of B. fragilis; and Bo-LPS represents the experimental group treated with lipopolysaccharide of B. ovatus.

FIG. 3B shows NF-κB activities of HEK-Blue-mTLR4 reporter cells induced by lipopolysaccharide of E. coli O111:B4 after being pre-treated with lipopolysaccharides of P. goldsteinii, P. distasonis, P. merdae, B. fragilis, or B. ovatus; wherein, “-” represents the control group pre-treated with PBS solution only; Pg-LPS represents the experimental group pre-treated with lipopolysaccharide of P. goldsteinii; Pd-LPS represents the experimental group pre-treated with lipopolysaccharide of P. distasonis; Pm-LPS represents the experimental group pre-treated with lipopolysaccharide of P. merdae; represents the experimental group pre-treated with lipopolysaccharide of B. fragilis; and Bo-LPS represents the experimental group pre-treated with lipopolysaccharide of B. ovatus.

FIG. 4A shows differentially expressed genes compared with the control group, which was without any lipopolysaccharide treatment, in dendritic cells after being treated with lipopolysaccharide of E. coli.

FIG. 4B shows g differentially expressed genes compared with the control group, which was without any lipopolysaccharide treatment, in dendritic cells after being treated with lipopolysaccharide of P. goldsteinii.

FIG. 4C shows biological process of differentially expressed genes compared with the control group, which was without any lipopolysaccharide treatment, in dendritic cells after being treated with lipopolysaccharide of E. coli.

FIG. 4D shows biological process of differentially expressed genes compared with the control group, which was without any lipopolysaccharide treatment, in dendritic cells after being treated with lipopolysaccharide of P. goldsteinii.

FIG. 5A shows grams of body weight loss of individuals with chronic obstructive pulmonary disease improved by lipopolysaccharide of P. goldsteinii according to one embodiment of the present invention.

FIG. 5B shows percentage of body weight loss of individuals with chronic obstructive pulmonary disease improved by lipopolysaccharide of P. goldsteinii according to one embodiment of the present invention.

FIG. 6A shows abnormality of forced vital capacity (FVC) of individuals with chronic obstructive pulmonary disease improved by lipopolysaccharide of P. goldsteinii according to one embodiment of the present invention.

FIG. 6B shows abnormality of functional residual capacity (FRC) of individuals with chronic obstructive pulmonary disease improved by lipopolysaccharide of P. goldsteinii according to one embodiment of the present invention.

FIG. 6C shows abnormality of chord compliance (Cchord) of individuals with chronic obstructive pulmonary disease improved by lipopolysaccharide of P. goldsteinii according to one embodiment of the present invention.

FIG. 6D shows abnormality of FEV100/FVC of individuals with chronic obstructive pulmonary disease improved by lipopolysaccharide of P. goldsteinii according to one embodiment of the present invention.

FIG. 7 shows infiltration of immune cell in lung of individuals with chronic obstructive pulmonary disease ameliorated by lipopolysaccharide of P. goldsteinii according to one embodiment of the present invention.

FIG. 8A is a histological image that emphysema of individuals with chronic obstructive pulmonary disease improved by lipopolysaccharide of P. goldsteinii according to one embodiment of the present invention.

FIG. 8B shows analysis results of the histological image that emphysema of individuals with chronic obstructive pulmonary disease improved by lipopolysaccharide of P. goldsteinii according to one embodiment of the present invention.

FIG. 9A shows results of IL-1β gene expression level in lung tissues of individuals with chronic obstructive pulmonary disease decreased by lipopolysaccharide of P. goldsteinii according to one embodiment of the present invention.

FIG. 9B shows results of TNF-α gene expression level in lung tissues of individuals with chronic obstructive pulmonary disease decreased by lipopolysaccharide of P. goldsteinii according to one embodiment of the present invention.

FIG. 9C shows results of IL-1β gene expression level in colon tissues of individuals with chronic obstructive pulmonary disease decreased by lipopolysaccharide of P. goldsteinii according to one embodiment of the present invention.

FIG. 9D shows results of TNF-α gene expression level in colon tissues of individuals with chronic obstructive pulmonary disease decreased by lipopolysaccharide of P. goldsteinii according to one embodiment of the present invention.

FIG. 10A shows amount of endotoxin in bronchoalveolar lavage fluids (BALF) of individuals with chronic obstructive pulmonary disease decreased by lipopolysaccharide of P. goldsteinii according to one embodiment of the present invention.

FIG. 10B shows amount of endotoxin in serum of individuals with chronic obstructive pulmonary disease decreased by lipopolysaccharide of P. goldsteinii according to one embodiment of the present invention.

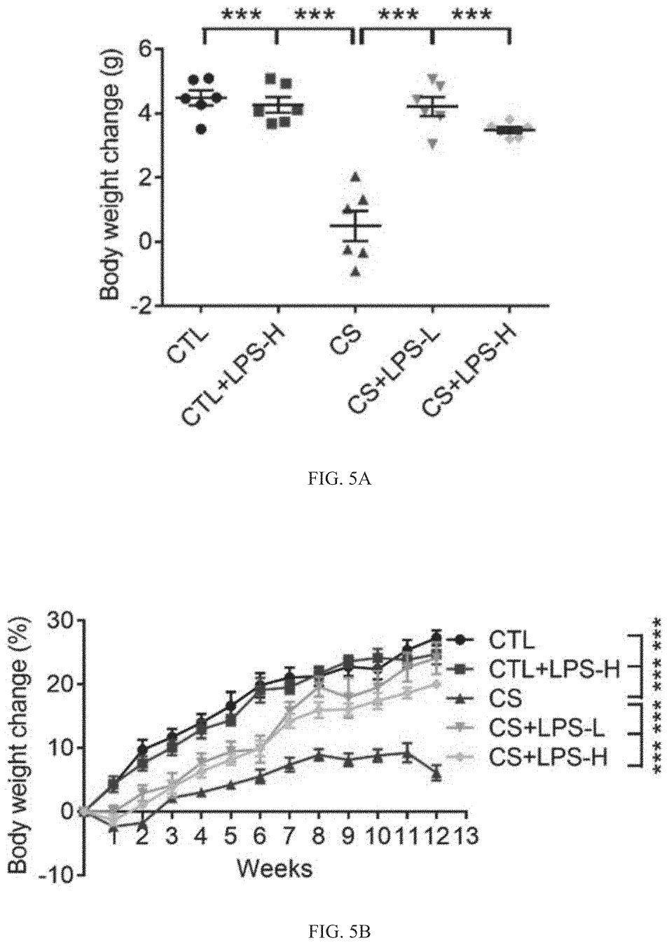

In FIGS. 5A to 10B above, CTL represents mice in the control group that were not treated with cigarette smoke or any lipopolysaccharide; CTL+LPS-H represents mice in the control group that were not treated with cigarette smoke but with high-dose of lipopolysaccharide of P. goldsteinii; CS represents mice in the comparative group that were treated with cigarette smoke but not with any lipopolysaccharide; CS+LPS-L represents mice in the experimental group that were treated with cigarette smoke and treated with low-dose lipopolysaccharide of P. goldsteinii; CS+LPS-H represents mice in the experimental group that were treated with cigarette smoke and high-dose lipopolysaccharide of P. goldsteinii.

FIG. 11 shows results of body weight gain of individuals inhibited by lipopolysaccharide of P. goldsteinii according to one embodiment of the present invention.

FIG. 12A shows results of glucose tolerance of individuals increased by lipopolysaccharide of P. goldsteinii according to one embodiment of the present invention.

FIG. 12B shows results of the area under the curve (AUC) of FIG. 12A.

FIG. 13A shows results of F4/80 gene expression level in intestinal tissues of individuals decreased by lipopolysaccharide of P. goldsteinii according to one embodiment of the present invention.

FIG. 13B shows results of MCP-1 gene expression level in intestinal tissues of individuals decreased by lipopolysaccharide of P. goldsteinii according to one embodiment of the present invention.

FIG. 13C shows results of IL-1β gene expression level in intestinal tissues of individuals decreased by lipopolysaccharide of P. goldsteinii according to one embodiment of the present invention.

FIG. 13D shows results of ZO-1 gene expression level in intestinal tissues of individuals increased by lipopolysaccharide of P. goldsteinii according to one embodiment of the present invention.

FIG. 13E shows results of Occludin gene expression level in intestinal tissues of individuals increased by lipopolysaccharide of P. goldsteinii according to one embodiment of the present invention.

FIG. 14 shows amount of endotoxin in serum of individuals with leaky gut syndrome decreased by lipopolysaccharide of P. goldsteinii according to one embodiment of the present invention.

In FIGS. 11 to 14 above, Chow represents mice in the control group that were fed with standard chow diet and not treated with any lipopolysaccharide; HFD represents mice in the control group that were fed with high-fat diet and not treated with any lipopolysaccharide; HFD+LPS represents mice in the experimental group that were fed with high-fat diet and treated with lipopolysaccharide of P. goldsteinii.

### DETAILED DESCRIPTION OF THE PREFERRED EMBODIMENT

All technical and scientific terms used herein, unless otherwise defined, have the meaning commonly understood by one with ordinary skill in the art.

Statistical analysis was performed using Excel software. Data were expressed as mean±standard deviation (SD) or mean±interquartile range (IQR), and Newman-Keuls multiple comparison post hoc one-way ANOVA was used to analyze whether the sample mean between each group was statistically significant.

The data provides in the present invention represent approximated, experimental values that vary within a range of ±20%, preferably ±10%, and most preferably ±5%.

As used herein, the term “hexa-acylated LPS” or “hexa-acylated lipopolysaccharide” refers to a lipopolysaccharide contains hexa-acylated lipid A structure, wherein the term “hexa-acylated lipid A structure” refers to the lipid A contains 6 fluorenyl chains.

As used herein, the term “hypo-acylated LPS” or “hypo-acylated lipopolysaccharide” refers to a lipopolysaccharide contains hypo-acylated lipid A structure, wherein the term “hypo-acylated lipid A structure” refers to the lipid A contains 1 to 5 fluorenyl chain(s).

As used herein, the term “intestinal integrity” refers to the integrity of the barrier function of an individual's intestinal tract, and more specifically refers to the tight connectivity of the individual's intestinal mucosal cells.

As used herein, the “disease associated with endotoxemia” includes but are not limit to: liver diseases, such as liver cirrhosis, primary biliary cholangitis and nonalcoholic fatty liver disease; metabolic syndrome, such as obesity and type II diabetes; inflammatory bowel diseases, such as active Crohn's disease and ulcerative colitis; pancreaticobiliary diseases, such as severe acute pancreatitis and obstructive jaundice; cardiorenal diseases, such as chronic heart failure and chronic kidney disease; psychological disorders, such as depression and autism; brain disorders, such as Alzheimer's disease/dementia, Parkinson disease and Huntington disease; skin diseases, such as psoriasis and atopic dermatitis; cancer; asthma; and ageing.

As used herein, the term “cancer” refers to all types of cancer or neoplasm or malignant tumors including leukemias, carcinomas and sarcomas, whether new or recurring. Specific examples of cancers include but are not limited to: carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma.

As used herein, the hypo-acylated LPS can be obtained by chemical synthesis, and can also be isolated and purified from bacteria, wherein the hypo-acylated LPS isolated and purified from bacteria of Bacteroidetes is preferably, and from bacteria of Bacteroides and Parabacteroide is more preferably. The bacteria of Bacteroides are preferably Bacteroides fragilis (B. fragilis), Bacteroides ovatus (B. ovatus), Bacteroides thetaiotaomicron (B. thetaiotaomicron), Bacteroides uniformis (B. umformis), Bacteroides vulgatus (B. vulgatus), and Bacteroides dorei (B. dorei); the bacteria of Parabacteroide are preferably Parabacteroides goldsteinii (P. goldsteinii), Parabacteroides distasonis (P. distasonis), and Parabacteroides merdae (P. merdae).

The hypo-acylated LPS of the present invention can be applied to a preparation of a pharmaceutical composition for improving anti-oxidation, preventing and/or treating endotoxemia, preventing and/or treating chronic obstructive pulmonary disease, preventing and/or treating obesity, and increasing glucose tolerance; wherein, the pharmaceutical composition may be a medicine, a nutritional supplement, a health food, or any combination thereof, and may further include a pharmaceutically acceptable excipient, carrier, adjuvant, and/or food additives.

In one preferred embodiment of the present invention, the hypo-acylated LPS of the present invention is formulated in a pharmaceutically acceptable vehicle, and is made into a suitable dosage form of an oral administration of, and the pharmaceutical composition is preferably in a dosage form selected from the following group: a solution, a suspension, a powder, a tablet, a pill, a syrup, a lozenge, a troche, a chewing gum, a capsule, and the like.

According to the present invention, the pharmaceutically acceptable vehicle may include one or more reagents selected from the following: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, and the like. The selection and quantity of these reagents is a matter of professionalism and routine for one with ordinary skill in the art.

According to the present invention, the pharmaceutically acceptable vehicle may include a solvent selected from the group consisting of: water, normal saline, phosphate buffered saline (PBS), aqueous solution containing alcohol, and combinations thereof.

In another preferred embodiment of the present invention, the hypo-acylated LPS of the present invention can be prepared into a food product, and be formulated with edible materials which include but not limited to: beverages, fermented foods, bakery products, health foods, nutritional supplements, and dietary supplements.

According to the present invention, the operating procedures and parameter conditions for bacterial culture are within the professional literacy and routine techniques of one with ordinary skill in the art.

According to the present invention, the operating procedures and parameter conditions for isolation and purification of lipopolysaccharide from bacteria are within the professional literacy and routine techniques of one with ordinary skill in the art.

According to the present invention, the operating procedures and parameter conditions for intraperitoneal injection in animals are within the professional literacy and routine techniques of one with ordinary skill in the art.

According to the present invention, the operating procedures and parameter conditions for buxco research systems in animals are within the professional literacy and routine techniques of one with ordinary skill in the art.

### Bacteria Cultivation of Bacteroides and Parabacteroides

Bacteroides and Parabacteroides are anaerobic bacteria and need to be cultured in an anaerobic incubator at 37° C. In the embodiments of the present invention, a Whitley DG250 anaerobic chamber (Don Whitley, Bingley, UK) was used to cultivate bacteria of Bacteroide and Parabacteroide, wherein the anaerobic chamber contains 5% carbon dioxide, 5% hydrogen, and 90% nitrogen, and an anaerobic indicator (Oxoid, Hampshire, UK) was used to confirm anaerobic conditions. The liquid culture medium of the bacteria is thioglycollate medium (BD, USA, #225710), and the solid medium is anaerobic blood agar (Ana. BAP) (Creative, New Taipei city, Taiwan). The bacteria can be stored in a refrigerator at −80° C. for a long-term preservation, and the protective liquid is 25% glycerin. There is no need for special cooling treatment, and can be stored by freeze-drying to stabilize its activity.

### LPS Purification

In the embodiments of the present invention, LPS were isolated from whole bacterial cells by using the hot phenol-water extraction. First, 1200 mL of bacterial culture solution cultured with the aforementioned method was centrifuged at 10000 g for 5 minutes, and then the supernatant was removed and the bacterial pellet was re-suspended in 30 mL of warm water and an equal volume of phenol was then added. The solution was stirred at 65° C. for 30 minutes, and then was centrifuged at 12000 g for 30 minutes to form a separated phase and the aqueous layers were collected. The organic layers were added an equal volume of warm water to perform the extraction twice to ensure that lipopolysaccharide in the mixture was completely collected. The aqueous layer solutions were combined and then subjected to dialysis and freeze-drying to obtain a crude extract of lipopolysaccharide. 0.1 mg/mL of deoxyribonuclease (DNase) and 0.1 mg/mL of ribonuclease (RNase) were added to treat the crude extract at 37° C. overnight, and then 0.05 mg/mL of proteolytic enzyme (e.g. Proteinase K) was added to treat the crude extract at 55° C. for 5 hours, and then further dialysis and freeze-drying to obtain a fluffy white solid which was lipopolysaccharide of each bacterium.

### Example 1

### Characteristic Analysis and Comparison of Lipopolysaccharides of Bacteroides and Parabacteroides

The lipopolysaccharides (LPS) of Gram-negative bacteria are mainly composed of three parts: lipid A, core oligo-saccharide, and O poly-saccharide (i.e. O antigen); wherein, lipid A is the main source of toxicity of lipopolysaccharide, and its main function is to assist lipopolysaccharide to fix on the cell membrane of bacteria. Thus, in one embodiment of the present invention, in order to analyze and compare the characteristics of lipopolysaccharides of Bacteroides and Parabacteroides, BLAST searched of the entire genome of six different Bacteroides and three different Parabacteroides were firstly performed to identify related genes responsible for biosynthesis of lipid A in lipopolysaccharide in these bacteria; wherein, Blast (Basic Local Alignment Search Tool) is an algorithm used to compare the primary structure of biological sequences (such as the amino acid sequences of different proteins or the DNA sequences of different genes). By comparing with information in a database known to contain several sequences, BLAST is a tool used to find existing sequences that are the same or similar to the sequence to be analyzed, in order to predict its efficacy or role. BLAST is based on KEGG and Search in NCBI-NR's data library.

In the embodiment of the present invention, the bacteria of Bacteroide selected for analysis include Bacteroides fragilis (B. fragilis), Bacteroides ovatus (B. ovatus), Bacteroides thetaiotaomicron (B. thetaiotaomicron), Bacteroides uniformis (B. umformis), Bacteroides vulgatus (B. vulgatus), and Bacteroides dorei (B. dorei); the bacteria of Parabacteroide selected for analysis include Parabacteroides goldsteinii (P. goldsteinii), Parabacteroides distasonis (P. distasonis), and Parabacteroides merdae (P. merdae); wherein, B. fragilis is NCTC9343 strain, B. ovatus is ATCC8483 strain, B. thetaiotaomicron is VPI-5482 strain, B. umformis is ATCC8492 strain, B. vulgatus is ATCC8482 strain, and B. dorei is DSM17855 strain; P. goldsteinii is DSM 32939 strain (patent deposit has been completed in US20200078414A1, referred to herein as MTS01 strain), P. distasonis is ATCC8503 strain, and P. merdae is ATCC43184 strain.

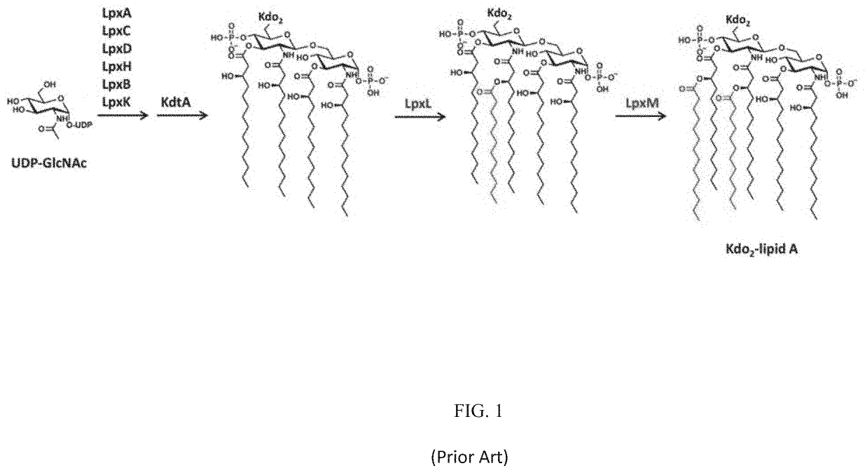

In the embodiment of the present invention, the BLAST search was based on Escherichia coli (E. coli) MG1655 strain (Genome accession number: U00096), and relevant genes responsible for biosynthesis of lipid A were used as a reference point for comparison; Lipid A of E. coli usually contains six fluorenyl chains (i.e. hexa-acylated structure), wherein, 3-deoxy-d-mannose-octanoic acid-lipid A (Kdo₂-lipid A) is the basic component of lipopolysaccharide in most gram-negative bacteria. As shown in FIG. 1 which is the biochemical synthesis pathway of Kdo₂-lipid A in E. coli, i.e. the Raetz pathway, uridine diphosphate N-acetylglucosamine (UDP-GlcNAc) is as the starting material of the reaction, and a total of seven enzymes LpxA, LpxC, LpxD, LpxH, LpxB, LpxK, and KdtA are used to synthesize the primary product of lipid A of E. coli. The fifth and sixth fluorenyl chains are added to the primary product through two enzymes, LpxL and LpxM, respectively, and lipid A of E. coli is completed, i.e. Kdo₂-lipid A in FIG. 1. Therefore, BLAST analysis was performed on nine genes related to Kdo₂-lipid A synthesis, i.e. LpxA, LpxC, LpxD, LpxH, LpxB, LpxK, KdtA, LpxL, and LpxM of E. coli.

The results of BLAST analysis and comparison were shown as Table 1. Among all the listed Bacteroides and Parabacteroides, the sequences of orthologous genes corresponding to LpxA, LpxC, LpxD, LpxH, LpxB, LpxK, KdtA, and LpxL could be found; however, there were no orthologous genes corresponding to LpxM can be found in the bacteria of Bacteroides or Parabacteroides. The analysis results indicate that the lipid A of lipopolysaccharide produced by bacteria of Bacteroides and Parabacteroides should only have five fluorenyl chains (i.e. penta-acylated structure) instead of six fluorenyl chains.

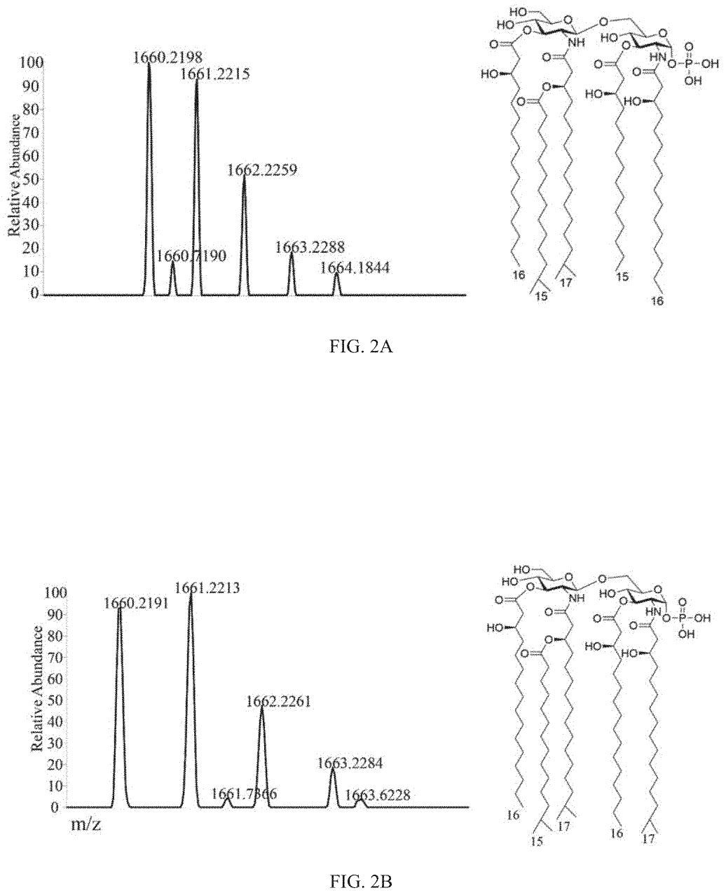

Therefore, in the embodiment of the present invention, the aforementioned method was further used to cultivate B. fragilis NCTC9343 and P. goldsteinii MTS01, and then lipopolysaccharides of these two bacteria were purified by the hot phenol-water extraction method, and structures of lipid A of the two lipopolysaccharides were analyzed by electrospray ionization coupled with mass spectrometry (ESI/MS). The analysis results of lipopolysaccharide of B. fragilis and P. goldsteinii were shown as FIG. 2A and FIG. 2B, respectively; wherein, the peaks of the detection signal were represented as the mass/charge ratio (m/z), and the predicted structures of lipid A related to the detected signal peak were displayed on the right side. As shown in FIGS. 2A and 2B, the lipopolysaccharides of B. fragilis and P. goldsteinii contained mass peaks with m/z of less than 1700. The results indicate that the two lipopolysaccharides contain the structure of hypo-acylated lipid A, more specifically, the m/z of the two was in the range of 1660.2 to 1664.2, indicating that the two lipopolysaccharides contain structure of penta-acylated lipid A.

According to the above BLAST analysis and ESI/MS analysis results, the lipopolysaccharides of Bacteroides and Parabacteroides actually provide hypo-acylated lipid A structures which have different structural characteristics from the pathogenic lipopolysaccharides of E. coli.

### Example 2

### Hypo-Acylated Lipopolysaccharides Provide Low Immune-Stimulatory Response and Low Endotoxicity

In one embodiment of the present invention, in order to further confirm whether lipopolysaccharides with hypo-acylated lipid A structure exhibit different endotoxicity and immune-stimulatory responses compared to lipopolysaccharides of E. coli, HEK-Blue-mTLR4 reporter cells (InvivoGen, U.S.), which are specifically used to measure the activity of pro-inflammatory lipopolysaccharides, were used to evaluate the ability of hypo-acylated lipopolysaccharides to activate the immune-stimulatory response of cells; wherein, the culture and determination steps of the HEK-Blue-mTLR4 reporter cells were performed in accordance with the manufacturer's operation manual.

According to the embodiment of the present invention, HEK-Blue-mTLR4 reporter cells were obtained by co-transfecting the co-receptor genes of mouse toll-like receptor 4 (TLR4), lymphocyte antigen 96 protein (also known as MD-2), and cluster of differentiation 14 (CD14) and the inducible secreted embryonic alkaline phosphatase (SEAP) reporter gene into HEK293 cells; wherein, SEAP was directly secreted into the culture medium of HEK-Blue-mTLR4 reporter cells, and the amount of SEAP in the culture medium could be estimated by the color change that SEAP hydrolyzes its substrate (i.e. HEK-Blue).

TABLE 1
BLAST analysis and comparison results of lipopolysaccharides

| | E. coli | P. goldsteinii | P. distasonis | P. merdae | B. fragilis | B. ovatus |
|---|---|---|---|---|---|---|
| gene | MG1655 | MTS01 | ATCC8503 | ATCC43184 | NCTC9343 | ATCC8483 |
| lpxA | b0180 | gene_109 | PD2124 | PM2041 | BF0827 | BACOVA_05052 |
| lpxC | b0096 | gene_110 | PD2123 | PM2042 | BF0828 | BACOVA_05053 |
| lpxD | b0179 | gene_111 | PD2122 | PM2043 | BF0829 | BACOVA_05054 |
| lpxH | b0524 | gene_4908 | PD2612 | PM1305 | BF0427 | BACOVA_03513 |
| lpxB | b0182 | gene_5050 | PD2656 | PM1387 | BF0699 | BACOVA_04823 |
| lpxK | b0915 | gene_160 | PD3985 | PM1717 | BF3273 | BACOVA_02939 |
| kdtA | b3633 | gene_5441 | PD2307 | PM1858 | BF4029 | BACOVA_01057 |
| lpxL | b1054 | gene_11 | PD0046 | PM0161 | BF3626 | BACOVA_03194 |
| lpxM | b1855 | — | — | — | — | — |

| | B. thetaiomicron | B. uniformis | B. vulgatus | B. dorei |
|---|---|---|---|---|
| gene | VPI-5482 | ATCC8492 | ATCC8482 | DSM17855 |
| lpxA | BT4205 | BACUNI_03478 | BVU_0099 | BACDOR_00466 |
| lpxC | BT4206 | BACUNI_03477 | BVU_0098 | BACDOR_00467 |
| lpxD | BT4207 | BACUNI_03476 | BVU_0097 | BACDOR_00468 |
| lpxH | BT3697 | BACUNI_00210 | BVU_0525 | BACDOR_01244 |
| lpxB | BT4004 | BACUNI_03661 | BVU_1917 | BACDOR_03148 |
| lpxK | BT1880 | BACUNI_02480 | BVU_1603 | BACDOR_02449 |
| kdtA | BT2747 | BACUNI_00837 | BVU_1476 | BACDOR_02423 |
| lpxL | BT2152 | BACUNI_03369 | BVU_1062 | BACDOR_01792 |
| lpxM | — | — | — | — |

In addition, in the HEK-Blue-mTLR4 reporter cells, five nuclear factor kappa-light-chain-enhancer of activated B cells (NF-κB) and AP-1 binding sites were fused with IFN-β minimal promoter to control the expression of the SEAP reporter gene. Therefore, when the TLR4 of HEK-Blue-mTLR4 reporter cells were stimulated by its ligand (the ligand referred to in the embodiment was lipopolysaccharides) to induce the expression of NF-κB and AP-1, the SEAP reporter gene expression would also be induced. Thus, by measuring the expression level of the SEAP reporter gene, the ability of lipopolysaccharide to promote the expression of NF-κB could be estimated, and then the ability of lipopolysaccharide to promote immune response of cells was evaluated.

In the embodiment of the present invention, the aforementioned method was firstly used to cultivate the P. goldsteinii MTS01, P. distasonis ATCC8503, P. merdae ATCC43184, B. fragilis NCTC9343, and B. ovatus ATCC8483, and then the lipopolysaccharides of these five bacteria were purified by hot phenol-water extraction method. The purified lipopolysaccharides of P. goldsteinii, P. distasonis, P. merdae, B. fragilis, and B. ovatus were prepared into two test solutions of 100 ng/mL and 1000 ng/mL with phosphate buffered saline solution (hereinafter referred to as PBS solution), respectively. The same method was used to prepare the comparison solutions of lipopolysaccharide of E. coli O111:B4 (Sigma, USA); wherein, E. coli O111:B4 is known as a pathogenic E. coli strain, and the lipopolysaccharide thereof can induce immune responses in cells of individuals.

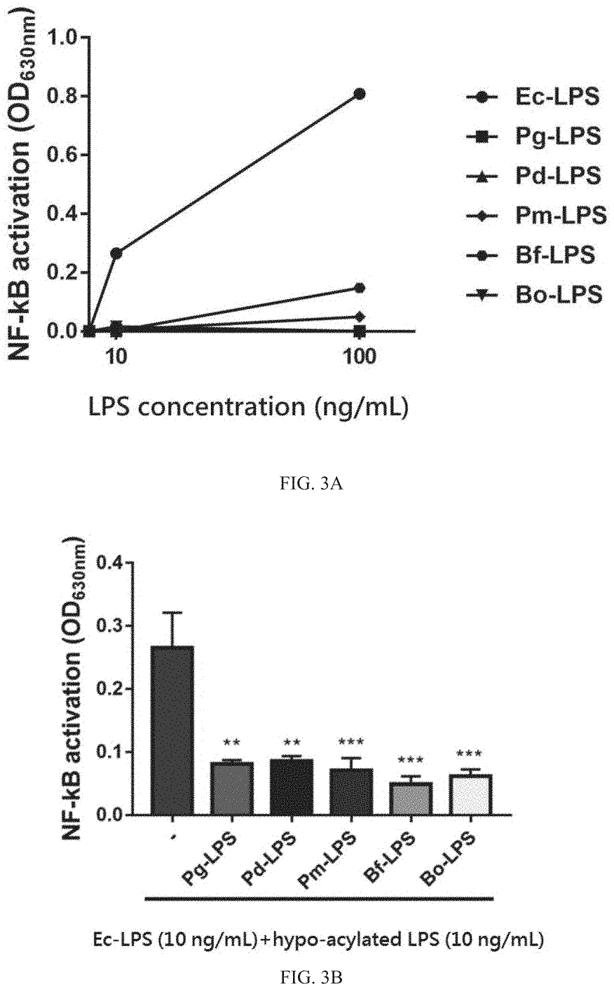

The previous test solutions and comparison solutions were separated into the following six groups: (1) the comparison group that was added with 10 μL of lipopolysaccharide of E. coli O111:B4 (represents as Ec-LPS); (2) the experimental group that was added with 10 μL of lipopolysaccharide of P. goldsteinii (represents as Pg-LPS); (3) the experimental group that was added with 10 μL of lipopolysaccharide of P. distasonis (represents as Pd-LPS); (4) the experimental group that was added with 10 μL of lipopolysaccharide of P. merdae (represents as Pm-LPS); (5) the experimental group that was added with 10 μL of lipopolysaccharide of B. fragilis (represents as Bf-LPS); and (6) the experimental group that was added with 10 μL of lipopolysaccharide of B. ovatus (represents as Bo-LPS). Then, the solution of the six groups were added into 90 μL of HEK-Blue-mTLR4 reporter cells (approximately 3×10⁴ cells), respectively, and the cells were cultured at 37° C. for 20 hours, and then 180 μL of the culture medium of each group of cells were taken out and added with L of Quanti-Blue (Invivogen) at 37° C. for 30 minutes. The absorbance of each group at OD630 were then measured to estimate the amount of SEAP in the culture medium of each group of cells, so as to observe the ability of lipopolysaccharides from P. goldsteinii, P. distasonis, P. merdae, B. fragilis, and B. ovatus to promote NF-κB expression, and then to evaluate the ability of hypo-glycated lipopolysaccharide to promote immune response of cells. The test results were shown as FIG. 3A.

FIG. 3A shows induced NF-κB activities of HEK-Blue-mTLR4 reporter cells after being treated with lipopolysaccharides of E. coli, P. goldsteinii, P. distasonis, P. merdae, B. fragilis, and B. ovatus. The lipopolysaccharide derived from E. coli O111:B4 could significantly induce activation of NF-κB at the concentration of 10 ng/mL, and at the concentration of 100 ng/mL, the lipopolysaccharide would more significantly induce the activation of NF-κB. On the contrary, the lipopolysaccharides derived from P. goldsteinii, P. distasonis, P. merdae, B. fragilis, and B. ovatus could not induce activation of NF-κB at the concentration of 10 ng/mL, and even the concentration was increased to 100 ng/mL, the hypo-acylated lipopolysaccharides could still not effectively induce activation of NF-κB. The results indicate that lipopolysaccharide derived from bacteria of Bacteroides or Parabacteroides hardly stimulates the activation of NF-κB in the HEK-Blue-mTLR4 reporter cells, that is, the type of lipopolysaccharide would not induce immune responses of cells, so hypo-acylated lipopolysaccharide provides low immune-stimulatory responses and low endotoxicity to individuals.

In the embodiments of the present invention, in order to further understand whether lipopolysaccharides with hypo-acylated lipid A structure could be used as an antagonist of pathogenic lipopolysaccharides to reduce the immune-stimulatory responses induced by the pathogenic lipopolysaccharides, the lipopolysaccharide of E. coli O111:B4 (Sigma, USA) was also prepared into a 200 ng/mL working solution with PBS solution, and the purified lipopolysaccharides of P. goldsteinii, P. distasonis, P. merdae, B. fragilis, and B. ovatus were also prepared into a 200 ng/mL test solution with PBS solution. Next, the previous test solutions were separated into the following six groups: (1) the control group that was added with only 5 μL of PBS solution; (2) the experimental group that was added with 5 μL of lipopolysaccharide of P. goldsteinii (represents as Pg-LPS); (3) the experimental group that was added with 5 L of lipopolysaccharide of P. distasonis (represents as Pd-LPS); (4) the experimental group that was added with 5 μL of lipopolysaccharide of P. merdae (represents as Pm-LPS); (5) the experimental group that was added with 5 μL of lipopolysaccharide of B. fragilis (represents as Bf-LPS); and (6) the experimental group that was added with 5 μL of lipopolysaccharide of B. ovatus (represents as Bo-LPS). Then, after the 90 μL of HEK-Blue-mTLR4 reporter cells (approximately 3×10⁴ cells) were pre-treated with the solution of the six groups, respectively, at 37° C. for 20 hours, the same amount of the working solution (i.e. the amount of lipopolysaccharide of E. coli O111:B4 and each hypo-acylated lipopolysaccharide was at a ratio of 1:1) was added for treating the cells at 37° C. for another 20 hours. Then, 180 μL of the culture medium of each group of cells were taken out and added with 20 μL of Quanti-Blue (Invivogen) at 37° C. for 30 minutes. The absorbance of each group at OD630 were then measured to estimate the amount of SEAP in the culture medium of each group of cells, so as to observe the antagonism of the lipopolysaccharides of P. goldsteinii, P. distasonis, P. merdae, B. fragilis, and B. ovatus to the expression of NF-κB induced by lipopolysaccharides of E. coli O111:B4, and then to evaluate the ability of hypo-glycated lipopolysaccharide to antagonize the immune-stimulatory responses of cells induced by pathogenic lipopolysaccharides. The test results were shown as FIG. 3B. The HEK-Blue-mTLR4 reporter cells that had not been pre-treated with any hypo-glycated lipopolysaccharide but had only been treated with pathogenic lipopolysaccharide were used as the control group.

FIG. 3B shows NF-κB activities of HEK-Blue-mTLR4 reporter cells induced by lipopolysaccharide of E. coli O111:B4 after being pre-treated with lipopolysaccharides of P. goldsteinii, P. distasonis, P. merdae, B. fragilis, and B. ovatus. Whether in the group that was pre-treated with lipopolysaccharide of P. goldsteinii, P. distasonis, P. merdae, B. fragilis, or B. ovatus, the NF-κB activity of HEK-Blue-mTLR4 reporter cells induced by the pathogenic lipopolysaccharide of E. coli O111:B4 would be significantly reduced to 20-30% of the control group that was without any pre-treatment. The results indicate that lipopolysaccharides derived from bacteria of Bacteroides or Parabacteroides can further antagonize the immune responses induced by the lipopolysaccharide of E. coli, and thus, the hypo-glycated lipopolysaccharides not only contain low immune-stimulatory responses, but also can antagonize the immune responses induced by pathogenic lipopolysaccharides.

### Example 3

### Hypo-Acylated Lipopolysaccharides Promote Cellular Antioxidant Responses

In one embodiment of the present invention, in order to further understand the direct effects or influence of hypo-glycated lipopolysaccharides on cells, transcriptomic analysis was performed on the cells treated with hypo-glycated lipopolysaccharides to understand the corresponding expression patterns in the cells and the key genes affected by the hypo-glycated lipopolysaccharides; wherein, transcriptome refers to the information of all RNA transcribed by the genome of the cell, and transcriptomic refers to the process of using high-throughput technology to observe the composition and abundance of the transcriptome in cells on a large scale.

First, the EasySep™ mouse CD11c positive selection kit (STEMCELL Technologies, Canada) was use to isolate the dendritic cells with the surface markers of cluster of differentiation 11 (CD11) from the mouse bone marrow cells stimulated by the granulocyte-macrophage colony-stimulating factor (GM-CSF). The dendritic cells were then cultured in 24-well plate with a cell content of 2×10⁵ per well, and the bone marrow derived dendritic cells (BMDCs) were separated into the following three groups: (1) the control group that was treated with only PBS solution at 37° C. for 4 hours; (2) the comparison group that was treated with 100 ng/mL of lipopolysaccharide of E. coli O111:B4 (Sigma, USA) at 37° C. for 4 hours (represents as Ec-LPS); and (3) the experimental group that was treated with 100 ng/mL lipopolysaccharide of P. goldsteinii MTS01 at 37° C. for 4 hours (represents as Pg-LPS). Next, the RNA extraction reagent kit (Geneaid, Taiwan) was used to extract total RNA in each group of dendritic cells for subsequent transcriptome analysis.

After the total RNA in each group of dendritic cells was extracted, Qubit® RNA Assay Kit (Life Technologies, California, USA) was firstly used with Qubit® 2.0 Fluorescence Detector (Life Technologies, California, USA) to check the quality of the total RNA, and the RNA Nano 6000 detection kit of Agilent Bioanalyzer 2100 system (Agilent Technologies, California, USA) was used to check the integrity of the total RNA. Then, cDNA library construction and Illumina sequencing were performed on the total RNA extracted from each group of dendritic cells to analyze the expression pattern of each gene in the dendritic cells and the key genes affected by pathogenic lipopolysaccharides or hypo-glycated lipopolysaccharides.

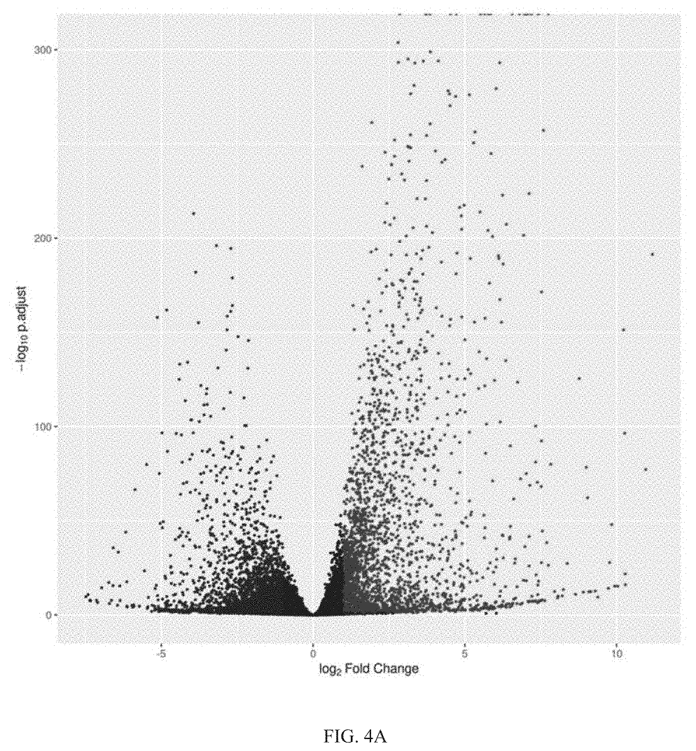

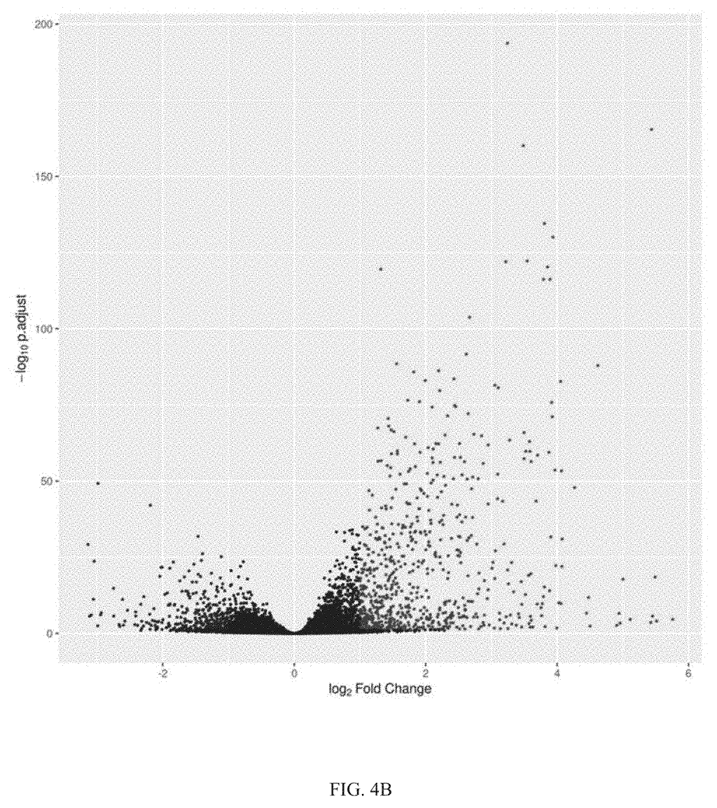

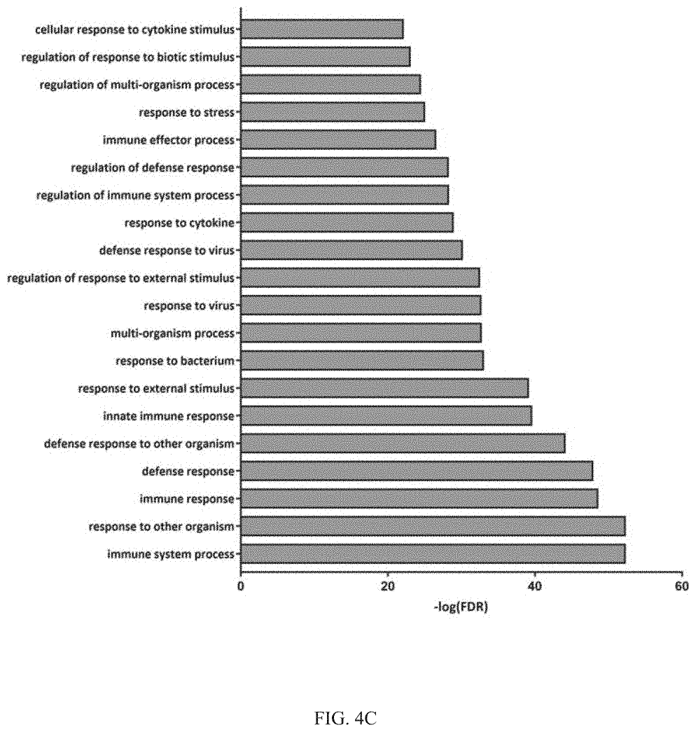

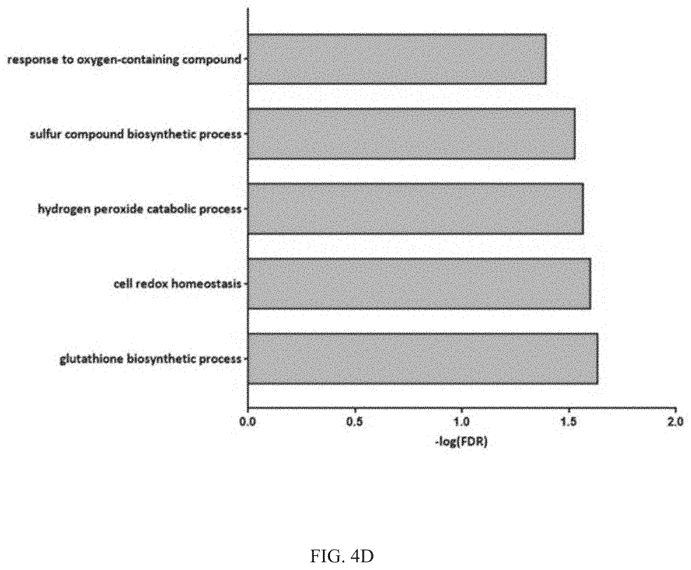

After the sequencing results of each gene in the total RNA of each group of dendritic cells were obtained, the software of RNA-Seq by Expectation-Maximization (RSEM) was used to quantify the expression level of each gene; wherein, the sequenced offline data (i.e. raw reads) was performed quality filtering to obtain the clean data (i.e. clean reads), and the clean data were mapped back onto the assembled transcriptome, and then the read count for each gene was obtained from the mapping results. Next, in order to further identify the key genes affected by pathogenic lipopolysaccharides and hypo-glycated lipopolysaccharides, DESeq was used to perform differential expression analysis to find out differentially expressed genes (DEGs) in the dendritic cells (1) treated with the lipopolysaccharide of E. coli O111:B4, or (2) treated with the lipopolysaccharide of P. goldsteinii comparing with the dendritic cells of the control group without any lipopolysaccharide treatment. The resulting p values were adjusted using Benjamini and Hochberg's approach for controlling the false discovery rate (FDR), and the genes with an adjusted p value <0.05 and |log 2 (fold change)|>1 were designated as significant DEGs. The analysis results of (1) and (2) were shown as volcano plots in FIGS. 4A and 4B, respectively. After the genes were classified to relate biological process (BP), the results of (1) were shown as FIG. 4C and Table 2, and the results of (2) were as shown as FIG. 4D and Table 3. DESeq was an R language package, which was used to analyze the expression level of each gene by counting reads per genes; and Gene Ontology (GO) enrichment of the DEGs was performed by using STRING databases.

FIG. 4C and Table 2 show biological process of differentially expressed genes compared with the control group, which was without any lipopolysaccharide treatment, in dendritic cells after being treated with lipopolysaccharide of E. coli O111:B4. The lipopolysaccharide of E. coli would upregulate biological process including immune system process, response to bacterium, and response to virus, which was indeed in line with the relevant cellular physiological responses caused by pathogenic lipopolysaccharides.

FIG. 4D and Table 3 show biological process of differentially expressed genes compared with the control group, which was without any lipopolysaccharide treatment, in dendritic cells after being treated with lipopolysaccharide of P. goldsteinii MTS01. The lipopolysaccharide of P. goldsteinii would upregulate biological process including glutathione biosynthetic process, cell redox homeostasis, hydrogen peroxide catabolic process, sulfur compound biosynthetic process, and response to oxygen-containing compound, which were all related to anti-oxidation. The results indicate that the lipopolysaccharide of P. goldsteinii could significantly improve the antioxidant capacity of cells, and thus lipopolysaccharides with the structure of hypo-acylated lipid A not only contained low immune-stimulatory responses, but also provided low endotoxicity to cells, and could further promote antioxidant responses of cells.

TABLE 2

| | | observed | background | | false |
|---|---|---|---|---|---|
| GO BP | | gene | gene | | discovery |
| term ID | term description | count | count | strength | rate |
| GO:0002376 | immune system process | 165 | 1703 | 0.62 | 5.94E−53 |
| GO:0051707 | response to other organism | 133 | 1050 | 0.74 | 5.94E−53 |
| GO:0006955 | immune response | 120 | 914 | 0.76 | 3.15E−49 |
| GO:0006952 | defense response | 128 | 1079 | 0.71 | 1.58E−48 |
| GO:0098542 | defense response to other organism | 104 | 735 | 0.79 | 9.02E−45 |
| GO:0045087 | innate immune response | 86 | 534 | 0.85 | 3.08E−40 |
| GO:0009605 | response to external stimulus | 158 | 2021 | 0.53 | 8.53E−40 |
| GO:0009617 | response to bacterium | 80 | 566 | 0.79 | 1.20E−33 |
| GO:0051704 | multi-organism process | 150 | 2092 | 0.49 | 2.40E−33 |
| GO:0009615 | response to virus | 55 | 221 | 1.03 | 2.50E−33 |
| GO:0032101 | regulation of response to external stimulus | 93 | 811 | 0.7 | 4.09E−33 |
| GO:0051607 | defense response to virus | 46 | 152 | 1.12 | 8.76E−31 |
| GO:0034097 | response to cytokine | 87 | 792 | 0.68 | 1.46E−29 |
| GO:0002682 | regulation of immune system process | 104 | 1165 | 0.59 | 6.35E−29 |
| GO:0031347 | regulation of defense response | 72 | 538 | 0.76 | 7.23E−29 |
| GO:0002252 | immune effector process | 61 | 395 | 0.83 | 3.69E−27 |
| GO:0006950 | response to stress | 164 | 2899 | 0.39 | 1.16E−25 |
| GO:0043900 | regulation of multi-organism process | 67 | 539 | 0.73 | 4.39E−25 |
| GO:0002831 | regulation of response to biotic stimulus | 51 | 308 | 0.86 | 1.03E−23 |
| GO:0071345 | cellular response to cytokine stimulus | 71 | 676 | 0.66 | 9.70E−23 |

TABLE 3

| | | observed | background | | false |
|---|---|---|---|---|---|
| GO BP | | gene | gene | | discovery |
| term ID | term description | count | count | strength | rate |
| GO:0006750 | glutathione biosynthetic process | 2 | 8 | 2.04 | 0.0233 |
| GO:0045454 | cell redox homeostasis | 3 | 63 | 1.32 | 0.0251 |
| GO:0042744 | hydrogen peroxide catabolic process | 2 | 14 | 1.8 | 0.0273 |
| GO:0044272 | sulfur compound biosynthetic process | 3 | 78 | 1.23 | 0.0296 |
| GO:1901700 | response to oxygen-containing compound | 10 | 1429 | 0.49 | 0.0407 |

### Example 4

### Hypo-Acylated Lipopolysaccharides Improve Chronic Obstructive Pulmonary Disease

Previous studies have shown that pathogenic lipopolysaccharides with a hexa-acylated lipid A structure could increase emphysema in patients with chronic obstructive pulmonary disease (COPD). Therefore, in one embodiment of the present invention, in order to further test the effects of lipopolysaccharide with hypo-acylated A structure on chronic obstructive pulmonary disease, mice with chronic obstructive pulmonary disease induced by cigarette smoke (CS) were used as animal model for experiments.

In the embodiment of the present invention, animal experiments were approved by the Institutional Animal Care and Use Protocol of Fu Jen Catholic University and were performed according to their guidelines. The experimental animals used herein were 8 to 10 week-old C57BL/6 mice which were purchased from the National Laboratory Animal Center (NLAC, Taipei, Taiwan) and kept under sterile conditions, following a 12-hour light/dark cycle, and were with one-week acclimatization period under this condition.

After the acclimatization period of experimental mice was over, the 8 to 10 week-old C57BL/6 mice were separated into the following five groups (n=6 in each group): (1) the control group (CTL): mice were exposed to indoor air only, and were injected 100 μL of PBS solution intraperitoneally at a frequency of twice a week for a total of 12 weeks; (2) the control group (CTL+LPS-H): mice were exposed to indoor air only, and were injected 100 μL of high-dose (100 μg/kg, about 2 g per mouse) of lipopolysaccharides isolated from P. goldsteinii MTS01 intraperitoneally at a frequency of twice a week for a total of 12 weeks; (3) the comparative group (CS): mice were exposed to the smoke of twelve 3R4F cigarettes (University of Kentucky) twice a day (that is, a total of twenty-four cigarettes per day) at a frequency of five times a week, and were injected 100 μL of PBS solution intraperitoneally at a frequency of twice a week for a total of 12 weeks; (4) the experimental group (CS+LPS-L): mice were exposed to the smoke of twelve 3R4F cigarettes (University of Kentucky) twice a day (that is, a total of twenty-four cigarettes per day) at a frequency of five times a week, and were injected 100 μL of low-dose (10 μg/kg, about 0.2 g per mouse) of lipopolysaccharides isolated from P. goldsteinii MTS01 intraperitoneally at a frequency of twice a week for a total of 12 weeks; and (5) the experimental group (CS+LPS-H): mice were exposed to the smoke of twelve 3R4F cigarettes (University of Kentucky) twice a day (that is, a total of twenty-four cigarettes per day) at a frequency of five times a week, and were injected 100 μL of high-dose (100 μg/kg, about 2 g per mouse) of lipopolysaccharides isolated from P. goldsteinii MTS01 intraperitoneally at a frequency of twice a week for a total of 12 weeks.

### 4-1 Hypo-Acylated Lipopolysaccharides Improve Body Weight Loss Caused by COPD

During the 12 weeks of the experimental duration, the body weight of each group of mice was monitored every week, and the starting body weight of the 0th week was subtracted from the final body weight of the 12th week as the value of weight gain, and the results were shown as FIG. 5A The body weight gain was divided by the starting body weight and expressed as a percentage to calculate the body weight change rate of each mouse in each group, and the results were shown as FIG. 5B. The data of the experimental results were expressed as mean±SD or mean±IQR, and Newman-Keuls multiple comparison post hoc one-way ANOVA was used for statistical analysis; wherein, * represents p value<0.05; ** represents p-value<0.01; and *** represents p-value<0.001.

As shown in FIG. 5A and FIG. 5B, compared with the mice in the control group (CTL) exposed to indoor air, the body weight gain of the mice in the comparison group (CS), in which COPD was induced by cigarette smoke, would significantly reduce, and the body weight change rate would also significantly reduce; however, after COPD was induced by cigarette smoke in the mice, compared with the comparison group (CS), the injection of the low-dose (CS+LPS-L) or high-dose (CS+LPS-H) of the hypo-acylated lipopolysaccharides of P. goldsteinii caused the body weight gain of the mice significantly increased to be equivalent to that of the control group (CTL), and the body weight change rate would also significantly increased by 13.9% and 18%, respectively. The results indicate that both low-dose and high-dose hypo-acylated lipopolysaccharide of P. goldsteinii can effectively alleviate the problem of body weight loss in individuals caused by COPD.

### 4-2 Hypo-Acylated Lipopolysaccharides Improve Abnormal Lung Function Caused by COPD

In the embodiment of the present invention, in order to further observe whether the hypo-acylated lipopolysaccharide of P. goldsteinii can improve the lung function of mice with COPD, after 12 weeks of the experiments in the aforementioned groups of mice, all mice were anesthetized, tracheostomized, and placed in the Buxco Research Systems (USA, hereinafter referred to as Buxco system) to evaluate lung functions. First, an average breathing frequency of 100 breaths/min was imposed to the anesthetized mice, and the Buxco system was used to perform three semi-automatic maneuvers, including the determination of functional residual capacity (FRC) by Boyle's law, quasistatic P-V, and fast flow volume maneuver; wherein, FRC was determined by Boyle's law; the operation for quasistatic P-V was to measure chord compliance (Cchord), and the operation for fast flow volume maneuver was to record forced expiratory volume (FEV), including the forced vital capacity (FVC) and the forced expiratory volume at the 100th millisecond (FEV100), and the operation for fast flow drive is to record the forced expiratory volume (Forced expiratory volume), FEV), including the forced vital capacity (FVC) and the forced expiratory volume at the 100th millisecond (FEV100).

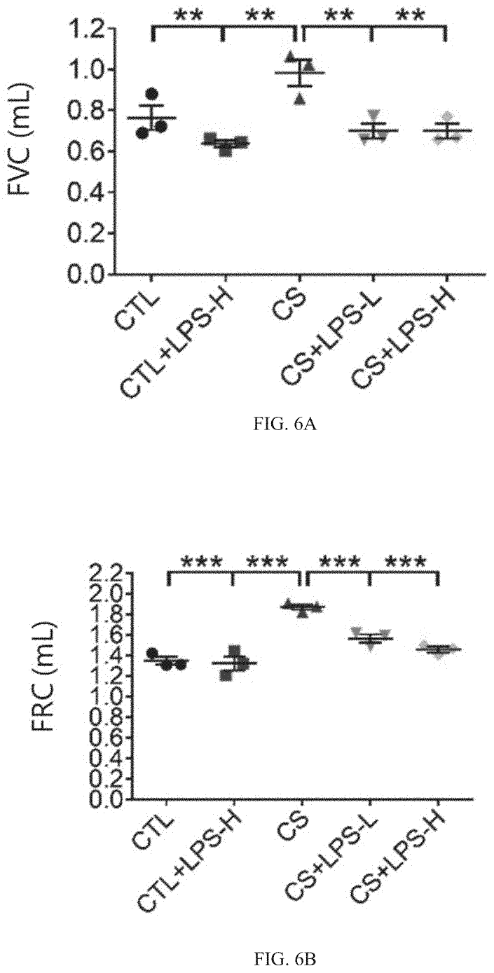

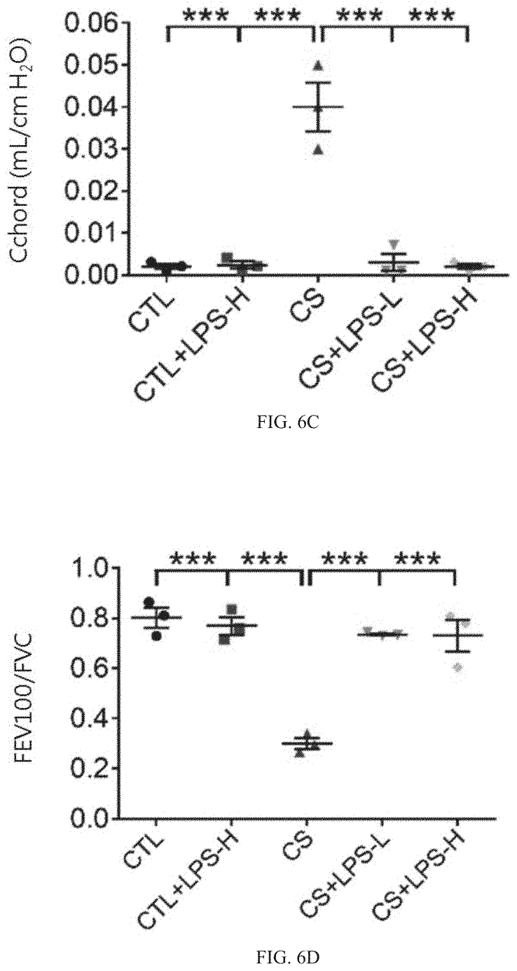

The test results of the lipopolysaccharide of P. goldsteinii improving abnormality of FVC of individuals with COPD were shown as FIG. 6A; the test results of the lipopolysaccharide of P. goldsteinii improving abnormality of FRC of individuals with COPD were shown as FIG. 6B; the test results of the lipopolysaccharide of P. goldsteinii improving abnormality of Cchord of individuals with COPD were shown as FIG. 6C; the test results of the lipopolysaccharide of P. goldsteinii improving abnormality of FEV100/FVC of individuals with COPD were shown as FIG. 6D. All the above maneuvers and perturbations were continuously performed until three correct measurements were achieved. The average of the three measurements of the above parameters for each mouse in each group was used as the result value for that parameter for that group of mice. The data of the experimental results were expressed as mean±IQR, and Newman-Keuls multiple comparison post hoc one-way ANOVA was used for statistical analysis; wherein, * represents p value<0.05; ** represents p-value<0.01; and *** represents p-value<0.001.

As shown in FIGS. 6A to 6D, compared with the mice in the control group (CTL) exposed to indoor air, the Cchord and FRC of the mice in the comparison group (CS), in which COPD was induced by cigarette smoke, significantly increased, indicating that the lung of the mice with emphysema induced by cigarette smoke had hyperinflation; furthermore, because the mice in the comparative group of (CS) had a larger lung volume during maximum inflation, the FVC thereof also significantly increased under forced exhalation; and the index of airflow obstruction during expiration, i.e. FEV100/FVC, of the mice in the comparison group of mice (CS) significantly decreased. The results indicate that the induction of COPD in mice with cigarette smoke would indeed reduce the lung function of the mice.

However, after COPD was induced by cigarette smoke in the mice, compared with the comparison group (CS), the injection of the low-dose (CS+LPS-L) or high-dose (CS+LPS-H) of the hypo-acylated lipopolysaccharides of P. goldsteinii caused the FVC, FRC, and Cchord of the mice significantly decreased to be equivalent to that of the control group (CTL), and the FEV100/FVC of the mice significantly increased to be equivalent to that of the control group (CTL). The results indicate that both low-dose and high-dose hypo-acylated lipopolysaccharide of P. goldsteinii can effectively improve the emphysema of individuals with COPD, and can effectively improve the lung function of individuals with COPD. 4-3 Hypo-acylated lipopolysaccharides ameliorate infiltration of immune cell in lung caused by COPD

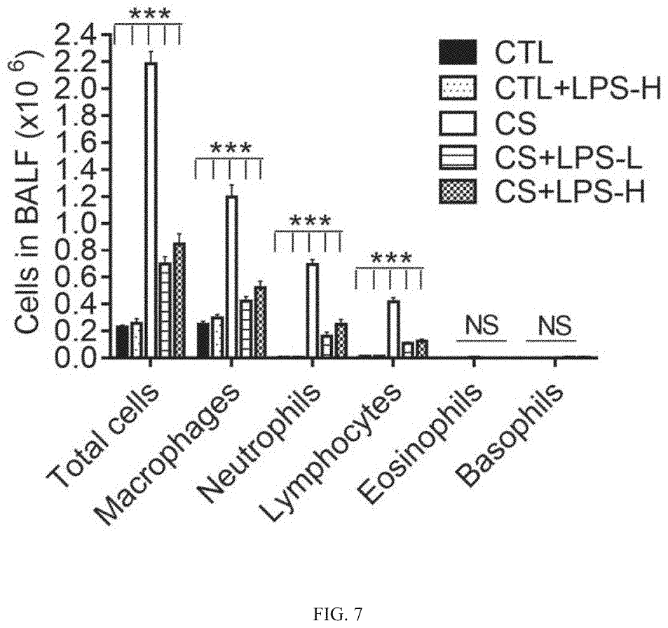

In the embodiments of the present invention, in order to further observe whether the hypo-acylated lipopolysaccharide of P. goldsteinii can ameliorate the infiltration of immune cell in lung of mice with COPD, the mice after 12 weeks of the experiments in the aforementioned groups were sacrificed, and the trachea of the mice was exposed by surgery, and a syringe was then inserted into the trachea to inject 800 L of PBS solution into the bronchus, and the bronchoalveolar lavage fluid (BALF) was aspirated out by the syringe, and then flow cytometry was used to analyze the amount of total cells, macrophage, neutrophil, lymphocytes, eosinophils, and basophil in the BALF of each group of mice, and the result were shown as FIG. 7. The data of the experimental results were expressed as mean±IQR, and Newman-Keuls multiple comparison post hoc one-way ANOVA was used for statistical analysis; wherein, * represents p value<0.05; ** represents p-value<0.01; and *** represents p-value<0.001.

As shown in FIG. 7, compared with the mice in the control group (CTL) exposed to indoor air, the amount of total cells, macrophage, and neutrophil in the BALF of the mice in the comparison group (CS), in which COPD was induced by cigarette smoke, would significantly increase, indicating that the mice in the comparison group had symptoms of chronic inflammation of the trachea, and with the additional involvement of lymphocytes which caused the inflammation persist and aggravate; however, after COPD was induced by cigarette smoke in the mice, compared with the comparison group (CS), the injection of the low-dose (CS+LPS-L) or high-dose (CS+LPS-H) of the hypo-acylated lipopolysaccharides of P. goldsteinii caused the amount of total cells, macrophage, neutrophil, and in the BALF of the mice significantly decreased. The results indicate that both low-dose and high-dose hypo-acylated lipopolysaccharide of P. goldsteinii can effectively ameliorate the infiltration of immune cell in lung of individuals with COPD.

### 4-4 Hypo-Acylated Lipopolysaccharides Improve Emphysema Caused by COPD

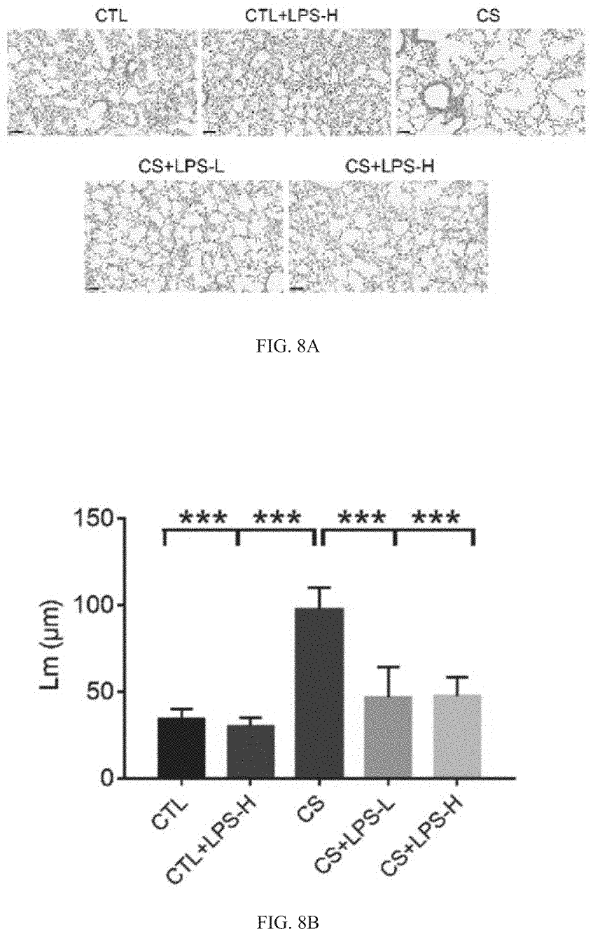

In the embodiment of the present invention, in order to more directly observe whether the hypo-acylated lipopolysaccharide of P. goldsteinii can improve the emphysema in mice with COPD, the mice after 12 weeks of the experiments in the aforementioned groups were sacrificed, and the lung tissues of each group of mice were taken out and fixed with formalin solution and then embedded in paraffin. The tissue sections with thickness of 4 mm were prepared and stained with hematoxylin and eosin (H&E). The stained sections were observed and recorded under an optical light microscope (Olympus, Tokyo, Japan), and the results were shown as FIG. 8A. The histological images were further analyzed by the ImageJ software (National Institutes of Health, Bethesda, USA) to determine the linear intercept (represents as Lm in FIG. 8B), wherein two randomly-selected fields from 10-15 sections of each group were analyzed, and the results were shown as FIG. 8B. The data of the experimental results were expressed as mean±SD, and Newman-Keuls multiple comparison post hoc one-way ANOVA was used for statistical analysis; wherein, * represents p value<0.05; ** represents p-value<0.01; and *** represents p-value<0.001.

As shown in FIGS. 8A and 8B, compared with the mice in the control group (CTL) exposed to indoor air, the alveolar wall of the mice in the comparison group (CS), in which COPD was induced by cigarette smoke, was damaged more seriously and the air gap of the alveolar was also enlarged, indicating that the mice in the comparison group had symptoms of emphysema; however, after COPD was induced by cigarette smoke in the mice, compared with the comparison group (CS), the injection of the low-dose (CS+LPS-L) or high-dose (CS+LPS-H) of the hypo-acylated lipopolysaccharides of P. goldsteinii caused the symptoms of emphysema of the mice decreased to be closer to that of the control group (CTL), and the mice injected with high-dose of the hypo-acylated lipopolysaccharides of P. goldsteinii showed almost normal lung patterns. The results indicate that both low-dose and high-dose hypo-acylated lipopolysaccharide of P. goldsteinii can effectively improve the emphysema of individuals with COPD.

### 4-5 Hypo-Acylated Lipopolysaccharides Ameliorate Secretion of Pro-Inflammatory Cytokines Caused by COPD

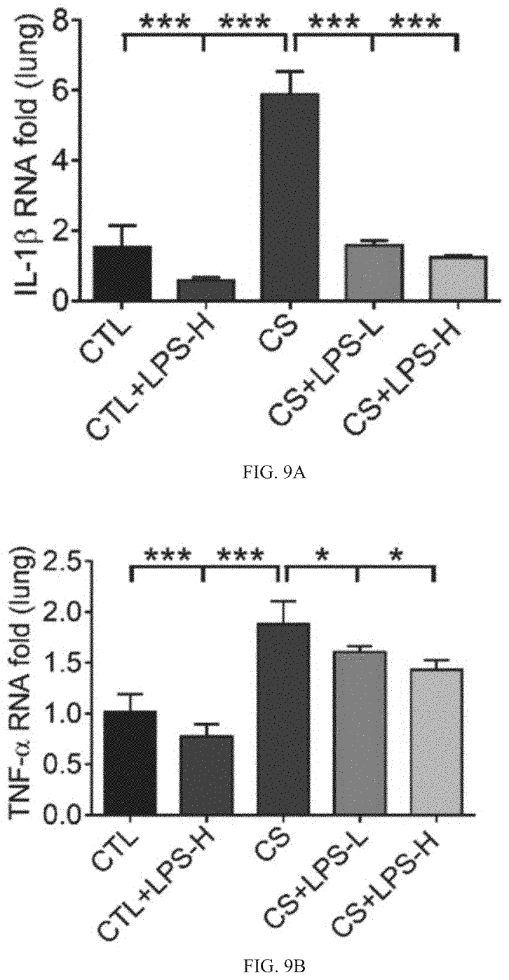

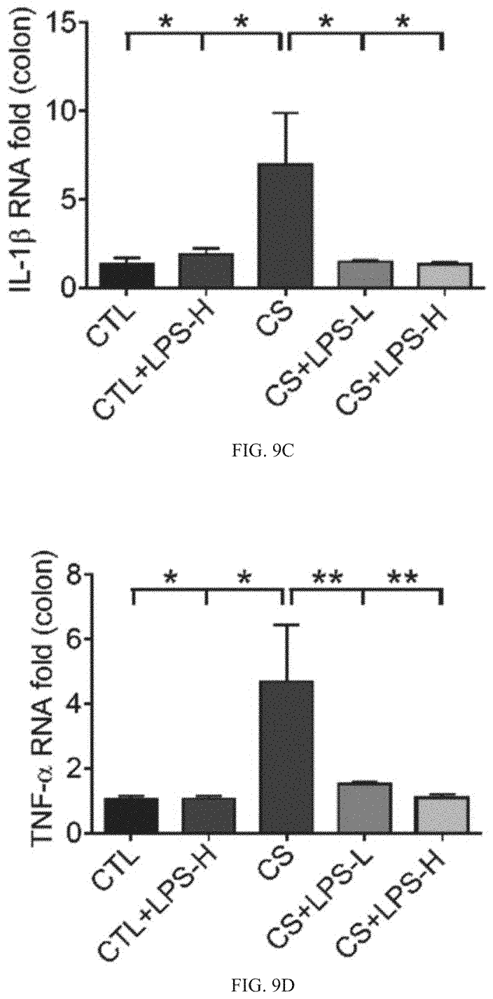

In the embodiment of the present invention, in order to further observe whether the hypo-acylated lipopolysaccharide of P. goldsteinii can directly ameliorate the expression and secretion of pro-inflammatory cytokines in mice with COPD, the mice after 12 weeks of the experiments in the aforementioned groups were sacrificed, and the lung tissues of each group of mice were collected. Then, RNeasy® MiniKit (Qiagen, Valencia, Calif., USA) was use to extract total RNA in the lung tissue cells, and the extracted total RNA was used as a template for reverse transcription by Quant II fast reverse transcriptase kit (Tools, Taipei, Taiwan) with random primers to produce the cDNA products corresponding to the mRNA of the specific genes. Then, 1 μL of the resulting cDNA was used as template and mixed well with 1 μL of target gene primers as shown in Table 4, 5 μL of 2× qPCRBIO SyGreen Blue Mix Lo-ROX (PCR Biosystems, London, UK) and 3 μL of double distilled water for performance of quantitative real-time polymerase chain reaction (qPCR) to detect the gene expression levels of tumor necrosis factor-α (TNF-α) and interleukin-1β (IL-1β) which were pro-inflammatory cytokines. The conditions of the qPCR were performed as described below: initial step of pre-incubation at 95° C. for 3 min, followed by 50 PCR cycles of 95° C. for 10 secs, 60° C. for 20 secs, 72° C. for 5 secs and then one melting curve cycle. Glyceraldehyde 3-phosphate dehydrogenase (GAPDH) was used as the internal control for qPCR assay. The results were shown as FIGS. 9A and 9B, and the data of the experimental results were expressed as mean±SD, and Newman-Keuls multiple comparison post hoc one-way ANOVA was used for statistical analysis; wherein, * represents p value<0.05; ** represents p-value<0.01; and *** represents p-value<0.001.

TABLE 4

| | | Sequence | |
|---|---|---|---|
| Gene | Primer | number | Sequence |
| GAPDH | GAPDH-F | SEQ ID NO: 1 | GCATCCACTGGTGCTGCC |
| | GAPDH-R | SEQ ID NO: 2 | TCATCATACTTGGCAGGTTTC |
| INF-α | TNF-α-F | SEQ ID NO: 3 | TAGCCAGGAGGGAGAACAGA |
| | TNF-α-R | SEQ ID NO: 4 | TTTTCTGGAGGGAGATGTGG |
| IL-1β | IL-1β-F | SEQ ID NO: 5 | TTGAAGAAGAGCCCATCCTC |
| | IL-1β-R | SEQ ID NO: 6 | CAGCTCATATGGGTCCGAC |

Furthermore, because cigarette smoke has also been confirmed as a risk factor for intestinal mucosal damage, the colon tissues of each group of mice were also collected. The same method was used to analyze the gene expression levels of TNF-α and IL-1β, which were pro-inflammatory cytokines, in the colon tissue cells. The results were shown as FIGS. 9C and 9D, and the data of the experimental results were expressed as mean±SD, and Newman-Keuls multiple comparison post hoc one-way ANOVA was used for statistical analysis; wherein, * represents p value<0.05; ** represents p-value<0.01; and *** represents p-value<0.001.

As shown in FIGS. 9A and 9B, compared with the mice in the control group (CTL) exposed to indoor air, both of the gene expression levels of TNF-α and IL-1β in the lung tissue cells of the mice in the comparison group (CS), in which COPD was induced by cigarette smoke, would significantly increase; however, after COPD was induced by cigarette smoke in the mice, compared with the comparison group (CS), the injection of the low-dose (CS+LPS-L) or high-dose (CS+LPS-H) of the hypo-acylated lipopolysaccharides of P. goldsteinii caused the gene expression levels of TNF-α and IL-1β in the lung tissue cells significantly decreased.

As shown in FIGS. 9C and 9D, compared with the mice in the control group (CTL) exposed to indoor air, both of the gene expression levels of TNF-α and IL-1β in the colon tissue cells of the mice in the comparison group (CS), in which COPD was induced by cigarette smoke, would significantly increase; however, after COPD was induced by cigarette smoke in the mice, compared with the comparison group (CS), the injection of the low-dose (CS+LPS-L) or high-dose (CS+LPS-H) of the hypo-acylated lipopolysaccharides of P. goldsteinii caused the gene expression levels of TNF-α and IL-1β in the colon tissue cells significantly decreased.

The results indicate that both low-dose and high-dose hypo-acylated lipopolysaccharide of P. goldsteinii can effectively ameliorate overexpression and secretion of pro-inflammatory cytokines in the lung tissues and even in the colon tissues of individuals with COPD, so as to effectively reduce inflammatory responses of the individuals.

### 4-6 Hypo-Acylated Lipopolysaccharides Reduce Circulating Endotoxin Levels Caused by COPD

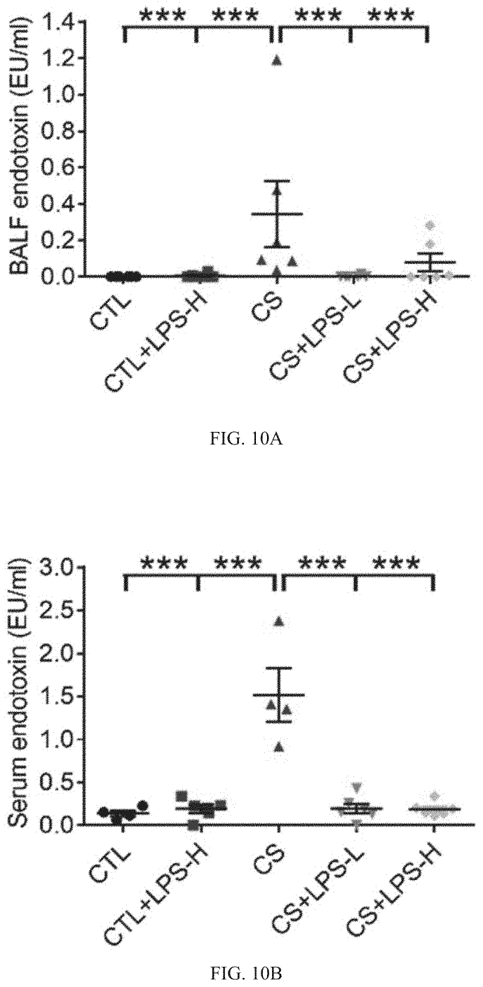

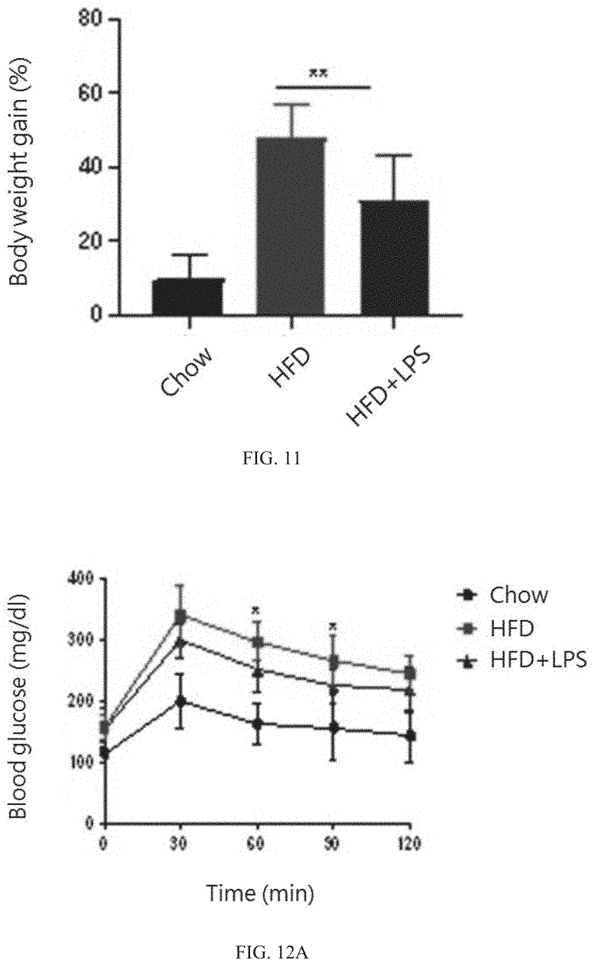

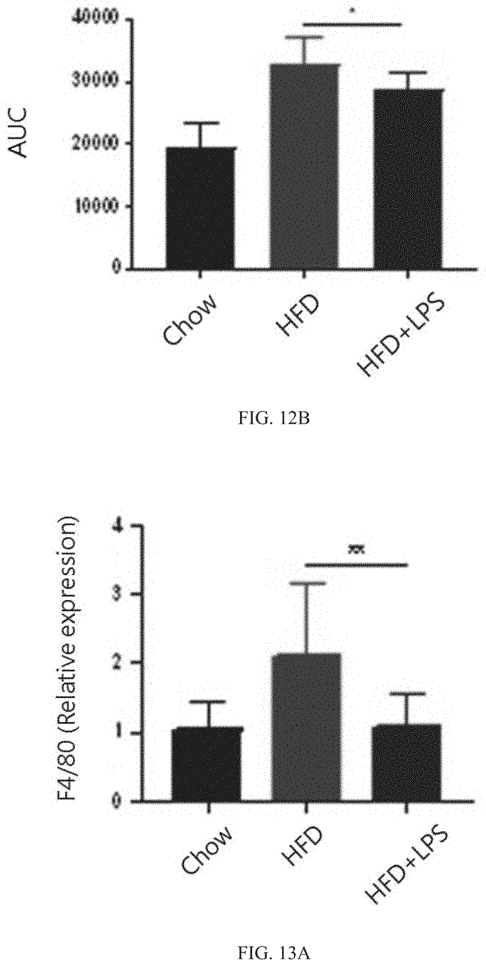

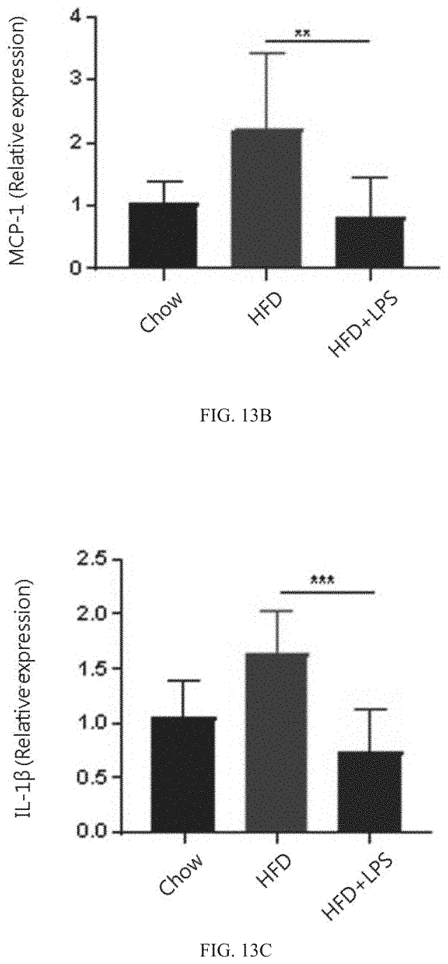

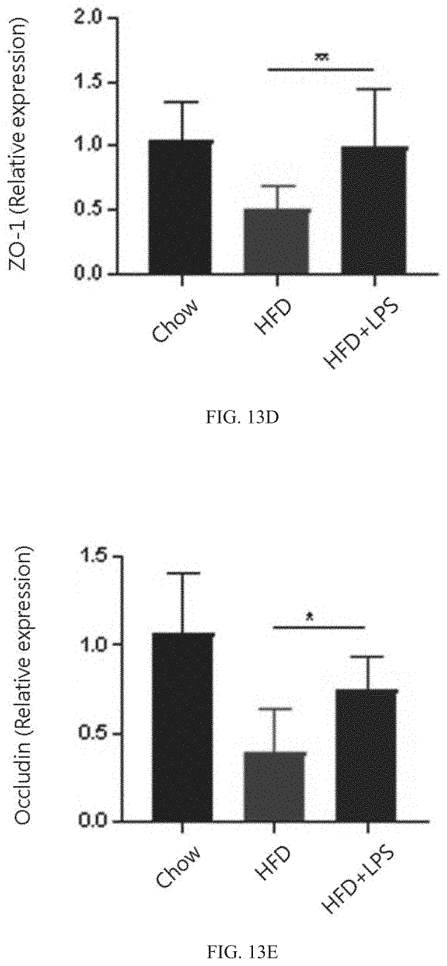

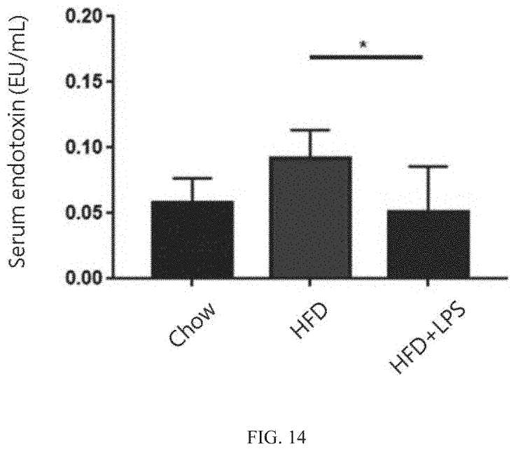

The increased amount of pathogenic lipopolysaccharides in the circulatory system of patients with COPD has been known to cause increases in oxidative stress and secretion of pro-inflammatory cytokines, and may also be related to the pathogenesis of COPD. Therefore, in the embodiment of the present invention, in order to further understand whether the hypo-acylated lipopolysaccharide can directly affect the amount of pathogenic lipopolysaccharides in the circulatory system of individuals, the mice after 12 weeks of the experiments in the aforementioned groups were sacrificed, and the BALF and serum were collected, and then the HEK-Blue-mTLR4 reporter cells (InvivoGen, USA) were used to detect and quantify the amount of pathogenic lipopolysaccharides (i.e. endotoxin) thereof. The results were shown as FIGS. 10A and 10B, and the operating procedures of the HEK-Blue-mTLR4 reporter cells were performed in accordance with the manufacturer's operation manual. The data of the experimental results were expressed as mean±IQR, and Newman-Keuls multiple comparison post hoc one-way ANOVA was used for statistical analysis; wherein, * represents p value<0.05; ** represents p-value<0.01; and *** represents p-value<0.001.

As shown in FIGS. 10A and 10B, compared with the mice in the control group (CTL) exposed to indoor air, the detected activity of lipopolysaccharide in both of BALF and serum of the mice in the comparison group (CS), in which COPD was induced by cigarette smoke, would significantly increase, indicating that COPD was indeed related to endotoxemia; however, after COPD was induced by cigarette smoke in the mice, compared with the comparison group (CS), the injection of the low-dose (CS+LPS-L) or high-dose (CS+LPS-H) of the hypo-acylated lipopolysaccharides of P. goldsteinii caused the detected activity of lipopolysaccharide in BALF and serum of the mice significantly decreased. The results indicate that both low-dose and high-dose hypo-acylated lipopolysaccharide of P. goldsteinii can effectively reduce the amount of pathogenic lipopolysaccharides in BALF and serum of individuals with COPD, and thus the lipopolysaccharides with hypo-acylated lipid A structure provide the effects of antagonizing and directly reducing the endotoxin in circulatory system of the individuals and can be used to improve endotoxemia.

In the embodiment of the present invention, compared with the pathogenic lipopolysaccharide with hexa-acylated lipid A structure, the lipopolysaccharides of the present invention with hypo-acylated lipid A structure have been proved that will not increase the severity of COPD while can effectively improve the symptoms of COPD and can effectively reduce the elevated endotoxin in blood of the individuals. The further experiments have shown that the mice treated with the hypo-acylated lipopolysaccharides of P. goldsteinii have normal liver and kidney functions (data not shown). Therefore, the hypo-acylated lipopolysaccharides derived from bacteria of Bacteroides or Parabacteroides provide the effects of preventing/treating COPD, and even the effects of preventing/treating endotoxemia.

### Example 5

### Hypo-Acylated Lipopolysaccharides Improve Obesity

In one embodiment of the present invention, in order to better understand the effects of lipopolysaccharides with hypo-acylated lipid A structure on diseases associated with endotoxemia, the obese mice induced by being fed with high-fat diets were used as animal model for experiments; wherein, the obesity induced by high-fat diets has been known to significantly increase the amount of endotoxin in blood of individuals.

In the embodiment of the present invention, animal experiments were approved by the Institutional Animal Care and Use Committee of Chang Gung University, and the experiments were performed in accordance with the guidelines. The experimental animals used herein were 6 week-old C57BL/6J male mice which were purchased from NLAC (Taipei, Taiwan) and were housed with free access to food and sterile drinking water in a temperature-controlled room (21±2° C.) under a 12-hour dark/light cycle, and were with one-week acclimatization period under this condition.

After the acclimatization period of experimental mice was over, the 6 week-old C57BL/6J male mice were separated into the following three groups (n=5 in each group): (1) the control group (Chow): mice were fed with standard chow diet (chow, 13.5% of energy from fat; LabDiet 5001; LabDiet, USA), and were injected 100 μL of PBS solution intraperitoneally at a frequency of twice a week for a total of 12 weeks; (2) the comparison group (high-fat diet, HFD): mice were fed with high-fat diet (HFD, 60% of energy from fat; TestDiet 58Y1; TestDiet, USA), and were injected 100 μL of PBS solution intraperitoneally at a frequency of twice a week for a total of 12 weeks; (3) the experimental group (HFD+LPS): mice were fed with high-fat diet, and were injected 100 μL of lipopolysaccharides isolated from P. goldsteinii MTS01 (100 μg/kg, about 2 g per mouse) intraperitoneally at a frequency of twice a week for a total of 12 weeks.

### 5-1 Hypo-Acylated Lipopolysaccharides Reduce Increases in Body Weight

After 12 weeks of the experiments in the aforementioned groups of mice, the starting body weight of the 0th week was subtracted from the final body weight of the 12th week as the value of body weight gain, and the body weight gain was divided by the starting body weight and expressed as a percentage to calculate the body weight change rate of each mouse in each group, and the results were shown as FIG. 11. Newman-Keuls multiple comparison post hoc one-way ANOVA was used for statistical analysis of the data of the experimental results; wherein, * represents p value<0.05; ** represents p-value<0.01; and *** represents p-value<0.001.

As shown in FIG. 11, compared with the mice in the control group fed with standard chow diet, the body weight change rate of the mice in the comparison group, in which obesity was induced by being fed with HFD, would significantly increase; however, when obesity was induced by being fed with HFD in the mice, compared with the comparison group, the further injection of the hypo-acylated lipopolysaccharides of P. goldsteinii caused the body weight change rate of the mice significantly decrease. The results indicate that the hypo-acylated lipopolysaccharide of P. goldsteinii can effectively reduce the body weight gain of individuals.

### 5-2 Hypo-Acylated Lipopolysaccharides Increase Glucose Tolerance

The increase of endotoxin in the blood has been known to promote the decrease in glucose tolerance of individual. Therefore, in the embodiment of the present invention, in order to further observe whether the hypo-acylated lipopolysaccharide of P. goldsteinii can reduce the impaired glucose tolerance of individuals, after 12 weeks of the experiments in the aforementioned groups of mice, the oral glucose tolerance test (OGTT) of the mice was performed. First, the mice in each group were given food for 8 hours, and the glucose solution (10%, w/v) was given to the mice by intragastric gavage at a dose of 1 g/kg, and the blood glucose of each group of mice was measured at 30-minute intervals before and after the gavage up to the 120 minutes. The results were shown as FIG. 12A. Area under the curve (AUC) values of each of the three groups of mice in FIG. 12A were calculated using the trapezoidal method and expressed as arbitrary units, and the results were shown as FIG. 12B. Newman-Keuls multiple comparison post hoc one-way ANOVA was used for statistical analysis of the data of the experimental results; wherein, * represents p value<0.05; ** represents p-value<0.01; and *** represents p-value<0.001.

As shown in FIGS. 12A and 12 B, compared with the mice in the control group fed with standard chow diet, after intragastric gavage of the glucose solution, the overall trend of the glucose concentration in the blood of mice in the comparison group, in which obesity was induced by being fed with HFD, would significantly increase over time, indicating that the glucose tolerance of the obesity mice in the comparison group significantly decreased; however, when obesity was induced by being fed with HFD in the mice, compared with the comparison group, the further injection of the hypo-acylated lipopolysaccharides of P. goldsteinii caused the overall trend of the glucose concentration in the blood of mice significantly decrease over time after intragastric gavage of the glucose solution, indicating that the glucose tolerance of the mice significantly increased. The results indicate that the hypo-acylated lipopolysaccharide of P. goldsteinii can effectively improve abnormal decreases in glucose tolerance of individuals.

### 5-3 Hypo-Acylated Lipopolysaccharides Promotes Intestinal Integrity and Reduces Intestinal Inflammation

As described above, the barrier dysfunction and high permeability of the intestine have been known to cause endotoxin translocate into the blood and then lead to endotoxemia and increase the risk of other diseases associated with endotoxemia. Therefore, in the embodiments of the present invention, in order to further understand whether lipopolysaccharides with hypo-acylated lipid A structure can more directly promote intestinal integrity and reduce intestinal inflammation of individuals, the mice after 12 weeks of the experiments in the aforementioned groups were sacrificed, and the intestinal tissue were collected. Then, RNeasy® MiniKit (Qiagen, Valencia, Calif., USA) was use to extract total RNA in the intestinal cells, and the extracted total RNA was used as a template for reverse transcription by Quant II fast reverse transcriptase kit (Tools, Taipei, Taiwan) with random primers to produce the cDNA products corresponding to the mRNA of the specific genes. Then, 1 μL of the resulting cDNA was used as template and mixed well with 1 μL of target gene primers as shown in Table 5, 5 μL of 2× qPCRBIO SyGreen Blue Mix Lo-ROX (PCR Biosystems, London, UK) and 3 μL of double distilled water for performance of quantitative real-time polymerase chain reaction (qPCR) to detect the gene expression levels of F4/80 (also known as adhesion G protein coupled receptors E1 (ADGRE1), or EMR1), monocyte chemoattractant protein-1 (MCP-1), IL-1β, zonula occludens-1 (ZO-1), and Occludin which were related to intestinal integrity or pro-inflammation. The conditions of the qPCR were performed as described below: initial step of pre-incubation at 95° C. for 3 min, followed by 50 PCR cycles of 95° C. for 10 secs, 60° C. for 20 secs, 72° C. for 5 secs and then one melting curve cycle. GAPDH was used as the internal control for qPCR assay.

TABLE 5

| | | Sequence | |
|---|---|---|---|
| Gene | Primer | number | Sequence |
| F4/80 | F4/80-F | SEQ ID | TTACGATGGAATTCTCCTTGTA |
| | | NO: 7 | TATCA |
| | F4/80-R | SEQ ID | CACAGCAGGAAGGTGGCTATG |
| | | NO: 8 | |
| MCP-1 | MCP-1-F | SEQ ID | CAGTCACGTGCTGTTATAATGT |
| | | NO: 9 | TGT |
| | MCP-1-R | SEQ ID | TATGGAATTCTTAACCCACTTC |
| | | NO: 10 | TCC |
| ZO-1 | ZO-1-F | SEQ ID | ACCCGAAACTGATGCTGTGGAT |
| | | NO: 11 | AG |
| | ZO-1-R | SEQ ID | AAATGGCCGGGCAGAACTTGTG |
| | | NO: 12 | TA |
| Occlu- | Occlu- | SEQ ID | ATGTCCGGCCGATGCTCTC |
| din | din-F | NO: 13 | |
| | Occlu- | SEQ ID | TTTGGCTGCTCTTGGGTCTGTA |
| | din-R | NO: 14 | T |

The results of gene expression levels of F4/80, MCP-1, and IL-1β in intestinal tissues of mice decreased by the lipopolysaccharide of P. goldsteinii were shown as FIGS. 13A, 13B, and 13C, respectively; the results of gene expression levels of ZO-1 and Occludin in intestinal tissues of mice increased by the lipopolysaccharide of P. goldsteinii were shown as FIGS. 13D and 13E, respectively. Newman-Keuls multiple comparison post hoc one-way ANOVA was used for statistical analysis of the data of the experimental results; wherein, * represents p value<0.05; ** represents p-value<0.01; and *** represents p-value<0.001.

As shown in FIGS. 13A to 13E, compared with the mice in the control group fed with standard chow diet, the gene expression levels of F4/80, MCP-1, and IL-1β in the intestinal tissue cells of the mice in the comparison group, in which obesity was induced by being fed with HFD, would significantly increase while the gene expression levels of ZO-1 and Occludin would significantly decrease, indicating that the obesity mice in the comparison group had lower intestinal integrity and also had symptoms of intestinal inflammation; however, when obesity was induced by being fed with HFD in the mice, compared with the comparison group, the further injection of the hypo-acylated lipopolysaccharides of P. goldsteinii caused the gene expression levels of F4/80, MCP-1, and IL-1β in the intestinal tissue cells of the mice significantly decreased to the equivalent to that of the control group, and caused the gene expression levels of ZO-1 and Occludin significantly increased to the equivalent to that of the control group, indicating that the intestinal integrity of the mice could be effectively restored and the symptoms of inflammation could also be effectively reduced. The results indicate that the hypo-acylated lipopolysaccharide of P. goldsteinii can effectively promote intestinal integrity and reduce intestinal inflammation of obesity individuals.

### 5-4 Hypo-Acylated Lipopolysaccharides Reduce Endotoxin Levels in Serum

In the embodiment of the present invention, in order to further observe whether lipopolysaccharide with hypo-acylated lipid A structure can directly reduce the amount of endotoxin in serum of individuals, the serum of the mice after 12 weeks of the experiments in the aforementioned groups were collected, and then the HEK-Blue-mTLR4 reporter cells (InvivoGen, USA) were used to detect and quantify the amount of pathogenic lipopolysaccharides (i.e. endotoxin) thereof. The results were shown as FIG. 14, and the operating procedures of the HEK-Blue-mTLR4 reporter cells were performed in accordance with the manufacturer's operation manual. Newman-Keuls multiple comparison post hoc one-way ANOVA was used for statistical analysis of the data of the experimental results; wherein, * represents p value<0.05; ** represents p-value<0.01; and *** represents p-value<0.001.

As shown in FIG. 14, compared with the mice in the control group fed with standard chow diet, the amount of endotoxin in the serum of the mice in the comparison group, in which obesity was induced by being fed with HFD, would significantly increase; however, when obesity was induced by being fed with HFD in the mice, compared with the comparison group, the further injection of the hypo-acylated lipopolysaccharides of P. goldsteinii caused the amount of endotoxin in the serum of the mice significantly decreased to the equivalent to that of the control group. The results indicate that the hypo-acylated lipopolysaccharide of P. goldsteinii can directly reduce the level of endotoxin in serum of individuals with risks of intestinal leakage, and can effectively improve endotoxemia of the individuals.

In the embodiment of the present invention, compared with the pathogenic lipopolysaccharide with hexa-acylated lipid A structure, the lipopolysaccharides of the present invention with hypo-acylated lipid A structure have been proved that can not only effectively reduce the body weight gain of individuals, but also can effectively improve abnormal decrease in glucose tolerance of individuals to prevent or treat obesity in individual, and can directly and effectively improve intestinal integrity and reduce intestinal inflammation of obesity individuals, and reduce the level of endotoxin in serum of individuals with risks of intestinal leakage. Therefore, the hypo-acylated lipopolysaccharide derived from bacteria of Bacteroides or Parabacteroides provides the effects of preventing and/or treating endotoxemia and reducing the risk of diseases associated with endotoxemia.

In summary, the present invention proves that the lipopolysaccharides with the structure of hypo-acylated lipid A contains low immune-stimulatory responses itself, and provides low endotoxicity to individuals, and can antagonize the immune responses induced by pathogenic lipopolysaccharides; moreover, the lipopolysaccharides with the structure of hypo-acylated lipid A can further promote antioxidant responses of cells, prevent and/or treat endotoxemia, and also prevent and/or treat diseases caused by pathogenic lipopolysaccharide or endotoxin, including but not limited to prevention/treatment of chronic obstructive pulmonary disease, prevention and/or treatment of obesity, and increasing of glucose tolerance.

What is claimed is:

1. A method for improving anti-oxidation, comprising administering to a subject in need thereof a composition comprising a hypo-acylated lipopolysaccharide, wherein a lipid A of the hypo-acylated lipopolysaccharide contains 1 to 5 fluorenyl chain(s).

2. The method according to claim 1, wherein the hypo-acylated lipopolysaccharide promotes glutathione biosynthetic process, cell redox homeostasis, hydrogen peroxide catabolic process, sulfur compound biosynthetic process, response to oxygen-containing compound, or any combination thereof.

3. The method according to claim 1, wherein an effective amount of the hypo-acylated lipopolysaccharide is 10 μg/kg for the subject in need thereof at least twice a week.

4. The method according to claim 1, wherein the hypo-acylated lipopolysaccharide is a hypo-acylated lipopolysaccharide from a bacterium of Bacteroidetes.

5. The method according to claim 4, wherein the hypo-acylated lipopolysaccharide is a hypo-acylated lipopolysaccharide from a bacterium of Bacteroide or Parabacteroide.

6. The method according to claim 1, wherein the composition further comprises a pharmaceutically acceptable excipient, carrier, adjuvant, or food additive.

7. The method according to claim 1, wherein the composition is in the form of a spray, a solution, a semi-solid preparation, a solid preparation, a gelatin capsule, a soft capsule, a tablet, a chewing gum, or a freeze-dried powder preparation.

8. A method of preventing and/or treating endotoxemia and a disease associated with endotoxemia, comprising administering to a subject in need thereof a composition comprising a hypo-acylated lipopolysaccharide, wherein a lipid A of the hypo-acylated lipopolysaccharide contains 1 to 5 fluorenyl chain(s).

9. The method according to claim 8, wherein the endotoxemia and the disease associated with endotoxemia is caused by leaky gut syndrome (LGS).

10. The method according to claim 8, wherein the hypo-acylated lipopolysaccharide promotes intestinal integrity of the subject in need thereof or reduces intestinal inflammation of the subject in need thereof.

11. The method according to claim 8, wherein the hypo-acylated lipopolysaccharide reduces the amount of endotoxin in blood of the subject in need thereof.

12. The method according to claim 8, wherein the disease associated with endotoxemia is selected from the group consisting of liver cirrhosis, primary biliary cholangitis, nonalcoholic fatty liver disease, obesity, type II diabetes, active Crohn's disease, ulcerative colitis, severe acute pancreatitis, obstructive jaundice, chronic heart failure, chronic kidney disease, chronic obstructive pulmonary disease, depression, autism, Alzheimer's disease/dementia, Parkinson disease, Huntington disease, psoriasis, atopic dermatitis, cancer, asthma, and ageing thereof.

13. The method according to claim 12, wherein the cancer is carcinoma, sarcoma, myeloma, leukemia, lymphoma, or mixed type tumor.

14. The method according to claim 8, wherein an effective amount of the hypo-acylated lipopolysaccharide is 10 μg/kg for the subject in need thereof at least twice a week.

15. The method according to claim 8, wherein the hypo-acylated lipopolysaccharide is a hypo-acylated lipopolysaccharide from a bacterium of Bacteroidetes.

16. The method according to claim 15, wherein the hypo-acylated lipopolysaccharide is a hypo-acylated lipopolysaccharide from a bacterium of Bacteroide or Parabacteroide.

17. The method according to claim 8, wherein the composition further comprises a pharmaceutically acceptable excipient, carrier, adjuvant, or food additive.

18. The method according to claim 8, wherein the composition is in the form of a spray, a solution, a semi-solid preparation, a solid preparation, a gelatin capsule, a soft capsule, a tablet, a chewing gum, or a freeze-dried powder preparation.

19. A method of preventing and/or treating chronic obstructive pulmonary disease, comprising administering to a subject in need thereof a composition comprising a hypo-acylated lipopolysaccharide, wherein a lipid A of the hypo-acylated lipopolysaccharide contains 1 to 5 fluorenyl chain(s).

20. The method according to claim 19, wherein the hypo-acylated lipopolysaccharide improves body weight loss, abnormal lung function, infiltration of immune cell in lung, emphysema, secretion of pro-inflammatory cytokines, or increase in circulating endotoxin levels caused by chronic obstructive pulmonary disease.

21. The method according to claim 20, wherein the pro-inflammatory cytokines includes tumor necrosis factor-α (TNF-α) or interleukin-1β (IL-1β).

22. The method according to claim 19, wherein an effective amount of the hypo-acylated lipopolysaccharide is 10 μg/kg for the subject in need thereof at least twice a week.

23. The method according to claim 19, wherein the hypo-acylated lipopolysaccharide is a hypo-acylated lipopolysaccharide from a bacterium of Bacteroidetes.

24. The method according to claim 23, wherein the hypo-acylated lipopolysaccharide is a hypo-acylated lipopolysaccharide from a bacterium of Bacteroide or Parabacteroide.

25. The method according to claim 19, wherein the composition further comprises a pharmaceutically acceptable excipient, carrier, adjuvant, or food additive.

26. The method according to claim 19, wherein the composition is in the form of a spray, a solution, a semi-solid preparation, a solid preparation, a gelatin capsule, a soft capsule, a tablet, a chewing gum, or a freeze-dried powder preparation.

27. A method of preventing and/or treating obesity, comprising administering to a subject in need thereof a composition comprising a hypo-acylated lipopolysaccharide, wherein a lipid A of the hypo-acylated lipopolysaccharide contains 1 to 5 fluorenyl chain(s).

28. The method according to claim 27, wherein the hypo-acylated lipopolysaccharide reduces increase in body weight of the subject in need thereof.

29. The method according to claim 27, wherein an effective amount of the hypo-acylated lipopolysaccharide is 10 μg/kg for the subject in need thereof at least twice a week.

30. The method according to claim 27, wherein the hypo-acylated lipopolysaccharide is a hypo-acylated lipopolysaccharide from a bacterium of Bacteroidetes.

31. The method according to claim 30, wherein the hypo-acylated lipopolysaccharide is a hypo-acylated lipopolysaccharide from a bacterium of Bacteroide or Parabacteroide.

32. The method according to claim 27, wherein the composition further comprises a pharmaceutically acceptable excipient, carrier, adjuvant, or food additive.

33. The method according to claim 27, wherein the composition is in the form of a spray, a solution, a semi-solid preparation, a solid preparation, a gelatin capsule, a soft capsule, a tablet, a chewing gum, or a freeze-dried powder preparation.

34. A method of increasing glucose tolerance, comprising administering to a subject in need thereof a composition comprising a hypo-acylated lipopolysaccharide, wherein a lipid A of the hypo-acylated lipopolysaccharide contains 1 to 5 fluorenyl chain(s).

35. The method according to claim 34, wherein an effective amount of the hypo-acylated lipopolysaccharide is 10 μg/kg for the subject in need thereof at least twice a week.

36. The method according to claim 34, wherein the hypo-acylated lipopolysaccharide is a hypo-acylated lipopolysaccharide from a bacterium of Bacteroidetes.

37. The method according to claim 36, wherein the hypo-acylated lipopolysaccharide is a hypo-acylated lipopolysaccharide from a bacterium of Bacteroide or Parabacteroide.

38. The method according to claim 34, wherein the composition further comprises a pharmaceutically acceptable excipient, carrier, adjuvant, or food additive.

39. The method according to claim 34, wherein the composition is in the form of a spray, a solution, a semi-solid preparation, a solid preparation, a gelatin capsule, a soft capsule, a tablet, a chewing gum, or a freeze-dried powder preparation.
